(19) United States
(12) Patent Application Publication
(10) Pub. No.: US 2026/0137635 A1
(43) Pub. Date: May 21, 2026

(54) NOVEL FORMULATIONS OF EPINEPHRINE AND USES THEREOF

(71) Applicant: NANJING HAIWEI PHARMACEUTICAL TECHNOLOGIES CO., LTD., Nanjing (CN)

(72) Inventors: Likun WANG, Nanjing (CN); Haoru SHI, Nanjing (CN)

(21) Appl. No.: 19/122,237

(22) PCT Filed: Oct. 20, 2023
(86) PCT No.: PCT/CN2023/125707

§ 371 Date: Apr. 17, 2025

(30) Foreign Application Priority Data
Oct. 21, 2022 (WO) PCT/CN2022/126776

(51) Int. Cl. A61K 31/137; A61K 9/00; A61K 9/08; A61K 47/02; A61K 47/10; A61K 47/18

(52) U.S. Cl. CPC A61K 31/137; A61K 9/0043; A61K 9/08; A61K 47/02; A61K 47/10; A61K 47/183; A61K 47/186

(57) ABSTRACT

It relates to novel formulations of epinephrine that comprise polyoxymethylene alkyl ethers as a permeation enhancer, methods of administering the formulation, and uses thereof.

### FIELD OF INVENTION

The present disclosure relates to novel formulations of epinephrine that comprise polyoxymethylene alkyl ethers as a permeation enhancer, methods of administering the formulation, and uses thereof.

### CROSS REFERENCE TO RELATED APPLICATION

This application claims the priority to and benefits of International Application No. PCT/CN2022/126776, filed Oct. 21, 2022, which is incorporated herein by reference in its entirety.

### BACKGROUND

Epinephrine is a hormone and a neurotransmitter produced by adrenal gland. It can also be chemically synthesized. Epinephrine has been used as a drug to treat various conditions.

Specifically, epinephrine is the drug of choice for treating type I allergic reaction, including anaphylaxis. It is estimated that up to 2% of the population worldwide will experience anaphylaxis at some point in life with an increasing trend (Simons et al., The World Allergy Organization Journal, 4 (2): 13-37 (2006)). Anaphylax is a serious, potentially fatal allergic reaction and medical emergency that is rapid in onset and requires immediate medical treatment regardless of use of emergency medication on site (Sampson et al., J. Allergy and Clinical Immunology, 117 (2): 391-7; Tintinalli, Judith E., Emergency Medicine: A Comprehensive Study Guide (2010) New York: McGraw-Hill Companies. pp. 177-182. ISBN 978-0-07-148480-0). Therefore, epinephrine autoinjectors were developed to allow faster self-administration of epinephrine via intramuscular route under emergency conditions (Mylan Specialty L.P. “EPIPEN®-epinephrine injection, EPIPEN Jr®-epinephrine injection”. FDA Product Label. (Archived (PDF) from the original on 1 Feb. 2014, Retrieved 22 Jan. 2014). However, epinephrine autoinjectors have rather high probability to result in undesirable situations, such as subcutaneous injection, or intravenous injection in error, or in the wrong strength (Bilò, M. Beatrice., Anaphylaxis caused by Hymenoptera stings: from epidemiology to treatment, Allergy 66, pages 35-37 (2011)). Autoinjectors are also complicated in structure, in which mechanical malfunctions can occur from time to time, making life-saving drug not as reliable as expected. Moreover, patients (with or without trypanophobia) are reluctant to use autoinjectors in public, also tend to delay using autoinjectors until having severe conditions, resulting in delayed treatment and failing to effectively reverse the fast progress of type I allergic reaction.

In view of challenges for epinephrine autoinjectors, epinephrine delivery approach with better reliability and patience compliance is highly desirable. Nasal delivery is one of the promising delivery routes: several epinephrine nasal sprays are under development. However, current nasal delivery formulations are limited by poor absorption, nasal membrane damage, and undesirable chemical stability. For instance, even with the aid of a permeation enhancer, more than 3 times dose (0.3 mg v.s. 1 mg) is needed for current nasal spray formulation compared with intramuscular delivery (Australia Patent No. AU2019217643B2). It is well known that epinephrine has narrow therapeutic window, poor absorption, which is often associated with high absorption variability. Thus, when higher dose is used to fix poor absorption issue, overdose can occur for some patients having higher absorption rate, resulting in severe adverse effect such as cerebral hemorrhage, hemiplegia, subarachnoid hemorrhage, respiratory difficulty etc. Similar situations have been identified as major safety risk that need further improvement for another drug (Hayley B. Schultz, et al., Oral Formulation Strategies to Improve the Bioavailability and Mitigate the Food Effect of Abiraterone Acetate, International Journal of Pharmaceutics, Volume 577, 119069, 2020). Moreover, European Medicines Agency (EMA) clearly indicated that smaller early partial AUC values, especially AUC of the first 10 minutes after dosing, are critical for the efficacy of epinephrine nasal spray; Tₘₐₓ is also relevant and should be the same or smaller than that with the intramuscular (IM) or subcutaneous (SC) route for the same epinephrine dose; and Cₘₐₓ and total AUC are deemed the most relevant parameters for drug product safety; Neffy, an epinephrine nasal spray submitted new drug application (NDA) to EMA, was not approved due to smaller early partial AUC compared with IM route (Assessment Report of Neffy, EMA/204348/2022, Committee for Medicinal Products for Human Use, 25 Mar. 2022).

Additionally, it has been reported that severe nasal cavity membrane damages have occurred when both the permeation enhancer and epinephrine are dosed via nasal route (Bleske et al., Effect of Vehicle on the Nasal Absorption of Epinephrine during Cardiopulmonary Resuscitation, Pharmacotherapy, 16 (6), 1039-1045 (1996)). Furthermore, epinephrine is highly liable to oxidation, thus the stability of epinephrine formulations can be challenging. (G. B. West, Oxidation of Adrenaline in Alkaline Solution, British Journal of Pharmacology and Chemotherapy, Volume 2, Issue 2, p. 121-130, 1947). Hence, there is a need to develop nasal formulations of epinephrine with higher bioavailability, less damage to nasal cavity membrane, lower variability, faster absorption rate and sufficient chemical stability.

### SUMMARY

The present disclosure provides novel formulations of epinephrine that comprise polyoxymethylene alkyl ethers as a permeation enhancer, methods of administering the epinephrine formulation, and uses thereof. The formulations described herein can have higher bioavailability, faster absorption rate, better chemical stability, and do less damage to nasal cavity membrane.

In an aspect, the present disclosure provides a pharmaceutical composition, comprising epinephrine and a permeation enhancer in the formula: CH₃(CH₂)ₙ₋₁[OCH₂CH₂]ₘOH, wherein n is an integer selected from 10-16; m is an integer selected from 4-8.

In some embodiments, n is 12, and m is selected from 4, 7 and 8.

In some embodiments, n is 10, and m is 6.

In some embodiments, the concentration of the permeation enhancer in the pharmaceutical composition described herein ranges from 0.1% to 2.50% (v/v).

In some embodiments, the concentration of the permeation enhancer in the pharmaceutical composition described herein is 0.25% (v/v).

The pharmaceutical composition of any one of claims 1-5, wherein the pH value of the pharmaceutical composition is lower than 7.

The pharmaceutical composition of any one of claims 1-6, wherein the pH value of the pharmaceutical composition ranges from 4 to 6.

In some embodiments, the pharmaceutical composition is used for nasal administration, or is used in a nasal delivery device.

In some embodiments, the pharmaceutical composition further comprises at least one pharmaceutically acceptable excipient, wherein the pharmaceutically acceptable excipient is a pH adjustment reagent, an antioxidant, a preservative, or an osmotic pressure adjustment reagent.

In some embodiments, the antioxidant is selected from sodium hydrogen sulfite, sodium metabisulfite (SMB), propyl gallate (PG), sodium sulfite, ascorbic acid (VC), methionine, alpha lipoic acid, cysteine (CYS), D-α-tocopheryl polyethylene glycol succinate (vitamine E TPGS), butylated hydroxytoluene (BHT) and butyl hydroxyanisole (BHA).

In some embodiments, the concentration of epinephrine in the pharmaceutical composition ranges from 0.3% to 5% (w/v).

In some embodiments, the pharmaceutical composition has a dosage form of liquid or spray.

In some embodiments, the recovery rate of the pharmaceutical composition at pH4.0, 60° C. is no lower than 90% by weight at day 30.

In some embodiments, effective permeability coefficient (Pe) of the pharmaceutical composition in a PAMPA test is higher than 3×10⁻⁶ cm/s.

In some embodiments, the pharmaceutical composition described herein does not cause irreversible damage to the nasal mucosa.

In another aspect, the present disclosure provides a method comprising administering an effective amount of the pharmaceutical composition described herein to the subject.

In some embodiments, the method is used to treat a subject with allergic reaction, especially type I allergic reaction.

In some embodiments, the allergic reaction is selected from allergic asthma, allergic conjunctivitis, allergic rhinitis, anaphylaxis, angioedema, urticaria, eosinophilia, drug allergy, and food allergy.

In some embodiments, the pharmaceutical composition is administered to the subject via nasal delivery.

### BRIEF DESCRIPTION OF THE FIGURES

FIG. 1 shows epinephrine concentration in plasma versus time for 10 mg/ml epinephrine concentration (1 mg/kg dose) group.

FIG. 2 shows epinephrine concentration in plasma versus time for 3 mg/mL epinephrine concentration (3 mg/mL epinephrine concentration, which is much lower than clinically used, 0.3 mg/kg dose) group.

FIG. 3 shows epinephrine concentration in plasma at different time after dosing compositions with different epinephrine concentrations, permeation enhancer types and concentrations.

FIG. 4 shows epinephrine concentration in plasma at different time after dosing compositions with different epinephrine concentrations and permeation enhancer types.

FIG. 5 shows Cmax (μU/mL) for each sub-group of each composition dosing.

FIG. 6 shows AUC (min*μU/mL) for each sub-group of each composition dosing.

### DETAILED DESCRIPTION

### Definition

In the present disclosure, unless otherwise specified, the scientific and technical terms used herein have the meanings generally understood by a person skilled in the art. Accordingly, the terms defined herein are more fully described by reference to the Specification as a whole.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present disclosure also contemplates that in some embodiments of the disclosure, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

It is to be understood that this disclosure is not limited to the particular methodology, protocols, and reagents described, as these may vary, depending upon the context in which they are used by those of skills in the art.

“Systemic delivery,” as used herein, refers to delivery of a therapeutic product that can result in a broad exposure of an active agent within an organism. “Local delivery,” as used herein, refers to delivery of an active agent directly to a target site within an organism. Local delivery does not preclude a systemic pharmacological effect.

As used herein, the term “pharmaceutically acceptable” refers to a material, such as a carrier or diluent, which does not abrogate the biological activity or properties of a therapeutic compound, and is relatively nontoxic, i.e., the material may be administered to a subject without causing undesirable biological effects or interacting in a deleterious manner with any of the components of the composition in which it is contained. Pharmaceutically acceptable components include those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “effective amount” refers to an amount of a pharmaceutical composition which is sufficient to significantly and positively modify the symptoms and/or conditions to be treated (e.g., provide a positive clinical response). The effective amount of a pharmaceutical composition will vary with the particular condition being treated, the severity of the condition, the duration of treatment, the nature of concurrent therapy, the particular composition being employed, the particular pharmaceutically acceptable excipient(s) and/or carrier(s) utilized, and like factors with the knowledge and expertise of the attending physician.

As used herein, a “disease” or “disorder” refers to a condition in which treatment is needed and/or desired.

As used herein, the term “treat,” “treating,” or “treatment” refers to ameliorating a disease or disorder, e.g., slowing or arresting or reducing the development of the disease or disorder or reducing at least one of the clinical symptoms thereof. For example, in some embodiments, ameliorating a disease or disorder can include obtaining a beneficial or desired clinical result that includes, but is not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

As used herein, the term “subject” refer to an animal. For example, in some embodiments, the animal is a mammal. In some embodiments, the animals are humans, rodents, simians, felines, canines, equines, bovines, porcines, ovines, caprines, mammalian laboratory animals, mammalian farm animals, mammalian sport animals, or mammalian pets. The animal can be male or female and can be at any suitable age, including infant, juvenile, adolescent, adult, and geriatric. In some examples, an “individual” or “subject” refers to an animal in need of treatment for a disease or disorder. In some embodiments, the animal to receive the treatment can be a “patient,” designating the fact that the animal has been identified as having a disorder of relevance to the treatment or being at adequate risk of contracting the disorder. In some embodiments, the animal is a human, such as a human patient.

As used herein, “permeability” refers to the ability of a pharmaceutical composition to pass across a biological membrane. In some embodiments, the biological membrane is nasal mucosa. Permeability can be measured by different permeability models which are performed, for example, in situ, ex vivo, or in vitro. Some exemplary permeability models are discussed in a later section.

As used herein, the term “permeation enhancer” refers to an excipient included in a formulation to improve the permeability of an active pharmaceutical ingredient. “Permeation enhancer” is sometimes also called “absorption enhancer” or “penetration enhancer.” In some embodiments, the permeation enhancer promotes nasal mucosa permeability. In some embodiments, the permeation enhancer promotes paracellular passage. In some embodiments, the permeation enhancer promotes transcellular passage.

As used herein, the term “nasal administration” or “nasal delivery” refers to administering a pharmaceutical composition into the nose of a subject for either topical administration or systemic administration. “Nasal” and “intranasal” are used interchangeably in the present disclosure.

As used herein, the terms “allergic reaction,” “allergic reaction response,” “allergy,” “allergic response,” and “allergic reaction” are used interchangeably.

As used herein, the term “active ingredient” in the context of a pharmaceutical composition refers to any component that provides pharmacological activity or other direct effect in the diagnosis, cure, mitigation, treatment, or prevention of disease, or to affect the structure or any function of the body of the subjects. For the purpose of the present disclosure, the active ingredient of an epinephrine composition described herein is epinephrine.

As used herein, the term “therapeutic window” refers to the range of a drug's serum concentration within which a desired effect occurs, below which there is little effect, and above which excessive toxicity occurs.

### Overview

Epinephrine is a first-line treatment for type I allergic reaction. Type I allergic reaction is also known as an immediate allergic reaction and involves immunoglobulin E (IgE) mediated release of antibodies against the soluble antigen. This results in mast cell degranulation and release of histamine and other inflammatory mediators. Type I hypersensitivities include atopic diseases, which are an exaggerated IgE mediated immune responses (for example, rhinitis, conjunctivitis, and dermatitis), and allergic diseases, which are immune responses to foreign allergens (for example, anaphylaxis, urticaria, angioedema, and food and drug allergies). By binding to multiple receptors of cells, epinephrine helps to increase blood flow, relax lung muscles, and suppress chemical release that causes allergic reactions. At certain dosages and routes of administration of epinephrine, the α-adrenergic vasoconstrictive effects reverse peripheral vasodilation, which alleviates hypotension and reduces erythema, urticaria, and angioedema. The β-adrenergic properties of epinephrine cause bronchodilation, increase myocardial output and contractility, and suppress further mediator release from mast cells and basophils.

Patients need rapid administration of epinephrine when they have a Type I allergic reaction. However, intramuscular epinephrine administration may be significantly delayed due to, for example, patient's fear of needles, lack of training, and misunderstanding of the right timing of administration. Such delay leads to poor outcomes and potentially death. Moreover, intramuscular epinephrine administration can potentially lead to intravenous delivery or subcutaneous delivery by mistake, resulting in severe side effects and significantly longer time to take effect. Nasal administration overcomes these problems by providing a more accessible way for patients to administering epinephrine timely and easily.

Nasal administration is a non-invasive delivery route, in which the pharmaceutical composition is insufflated through the nose and absorbed through nasal mucosa. A drug should first pass through the nasal mucous layer and then the epithelial layer before absorbed to achieve systemic effect. A drug administered through the nasal cavity can permeate either passively by the paracellular pathway or both passively and actively via the transcellular pathway. This process is largely influenced by lipophilicity of the compound. Apart from the passive transport pathways, carrier mediated transport, transcytosis and transport through intercellular tight junctions are other possible pathways for a drug to permeate across the nasal mucosa. Arora et al., Permeability Issues in Nasal Drug Delivery, Drug Discovery Today Vol. 7-18, 2002. For a polar and hydrophilic drug like epinephrine, the main pathway is paracellular passage, which is related to the intercellular cavity and tight junctions.

There are two main barriers for drug absorption in nasal administration, which are low membrane permeability of polar drugs, and rapid mucociliary clearance in the nasal cavity. Therefore, in order to achieve a comparable systemic absorption and pharmacokinetics of intramuscular injection, it is important to find a more desirable permeation enhancer for the nasal formulation. The present disclosure provides such effective permeation enhancers and uses thereof.

### Permeation Enhancer

Polyoxyethylene alkyl ethers are non-ionic surfactants made of a linear alkyl chain with n-1 methylene groups and a hydrophilic part with m oxyethylene units. They have the general formula CₙH₂ₙ₊₁(OCH₂CH₂)ₘOH (formula I). They are also referred to as CnEm, with n indicating the number of carbons in the alkyl chain and m being the number of ethylene oxide units in the hydrophilic moiety.

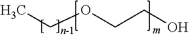

Although polyoxyethlene alkyl ethers have been nominated for permeation enhancer in previous publications, polyoxyethlene alkyl ether is a huge family which encompasses an indefinite number of compounds, few of them have been used effectively and safely as permeation enhancer in any commercially available nasal formulation. For example, polyoxyethylene-9-lauryl ether (C12E9) has been reported to cause severe multifocal erosion of the nasal epithelium in dog, when administered intranasally at a concentration of 1%, suggesting polyoxyethlene alkyl ethers might not be suitable for epinephrine nasal delivery (Bleske et al., Effect of Vehicle on the Nasal Absorption of Epinephrine During Cardiopulmonary Resuscitation, Pharmacotherapy 1996; 16 (6): 1039-1045). However, current work surprisingly found particular types of polyoxyethlene alkyl ethers showed significantly higher bioavailability, shorter onset time, better safety profiles and chemical stability compared with other formulations, which using C12E9 and other types of permeation enhancers, for epinephrine nasal administration.

In an aspect, the present disclosure provides a pharmaceutical composition, comprising epinephrine and a permeation enhancer in the formula: CH₃(CH₂)ₙ₋₁[OCH₂CH₂]ₘOH, wherein n is an integer selected from 10, 11, 12, 13, 14, 15, and 16; and m is an integer selected from 4, 5, 6, 7, and 8.

In some embodiments, n is 12, and m is selected from 4, 7, and 8.

In some embodiments, n is 10, and m is 6.

For the purpose of this disclosure, epinephrine encompasses both free form epinephrine and pharmaceutically acceptable salt of epinephrine, which includes acid and base addition salts.

“Pharmaceutically acceptable acid addition salt” refers to those salts which retain the biological effectiveness and properties of the free bases, which are not biologically or otherwise undesirable, and which are formed with inorganic acids or organic salts. Some exemplary acids are hydrochloric acid, tartaric acid, hydrobromic acid, sulfuric acid, nitric acid, phosphoric acid and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like.

The disclosure provided herein is also meant to encompass all pharmaceutically acceptable compounds as described herein being isotopically-labelled by having one or more atoms replaced by an atom having a different atomic mass or mass number. Examples of isotopes that can be incorporated into the disclosed compounds include isotopes of hydrogen, carbon, and oxygen, such as 2H, 3H, 11C, 13C, 14C, 15O, 17O, and 18O.

In some embodiments, the concentration of the permeation enhancer ranges from 0.1% to 2.50% (v/v). In some embodiments, the concentration of the permeation enhancer is 0.25% (v/v). In some embodiments, the concentration of the permeation enhancer is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% (v/v).

As used herein, “w/v” refers to weight per volume, also known as mass per volume. A concentration in weight per volume is calculated by dividing the mass of solute by volume of solution; grams of solute per 100 mL of solution (g/100 mL) was employed throughout current disclosure. As used herein, “v/v” refers to volume per volume. A concentration in volume per volume is calculated by dividing the volume of solute by volume of solution. For the purpose of the present disclosure, when a concentration is written as “w/v or v/v,” it means that the concentration is w/v for a solid solute, and the concentration is v/v for a liquid solute. For a compound that is solid at ambient temperature but has a low melting point, e.g., C12E8, its v/v concentration is obtained by melting the compound first before making the solution.

It is understood that a person skilled in the art is able to convert w/v to v/v, and vice versa, given the density of the solute compound. The density of C10E6 is about 0.987 g/mL. The density of C12E4 is about 0.946 g/mL. The density of C12E7 is about 1.0 g/mL. The density of C12E8 is about 0.984 g/mL (measured under 35° C.). The density of C12E9 is about 1.007 g/mL.

In some embodiments, the pharmaceutical composition is used for nasal administration, or is used in a nasal delivery device. In some embodiments, nasal administration is carried out by directly applying a pharmaceutical composition to the nasal mucosa. In some embodiments, nasal administration is carried out by a subject by snorting a pharmaceutical composition into the nasal cavity.

Nasal cavity is divided into vestibule, atrium, inferior turbinate, middle turbinate, and superior turbinate. Drug deposition following intranasal administration mainly occurs in the respiratory zone around the inferior turbinate (Grassin-Delyle et al., Pharmacology & Therapeutics 134:366-379 (2012)). Nasal mucosa comprises a layer of epithelium cells covering the nasal cavity. Drugs can be absorbed into the systemic circulation through the nasal mucosa.

Some exemplary nasal delivery devices are vapor inhaler, dropper, pipette, squeeze bottle, spray pump, nebulizer, powder spray, and insufflator (Djupesland, Drug Deliv. And Transl. Res. 3:42-62, 2013). It is understood that a person skilled in the art can choose a suitable nasal delivery device for a given pharmaceutical composition according to its dosage form, chemical properties, physical properties, and other relevant considerations.

In some embodiments, the concentration of epinephrine in the pharmaceutical composition described herein ranges from 0.3% to 5% (w/v). In some embodiments, the concentration of epinephrine in the pharmaceutical composition described herein is about 0.3%, 0.5%, 1%, 1.5%, 2%, 2.5%, 3%, 3.5%, 4%, 4.5%, or 5% (w/v).

In some embodiments, the pharmaceutical composition described herein has a dosage form of liquid. In some embodiments, the pharmaceutical composition described herein has a dosage form of spray. In some embodiments, the pharmaceutical composition described forms aqueous droplets.

### Permeability

As used herein, “permeability” refers to the ability of a pharmaceutical composition to pass across a biological membrane. For the purpose of this disclosure, effective permeability coefficient (Pe) in the Parallel Artificial Membrane Permeability Assay (PAMPA) is used as an index for permeability test in vitro. The PAMPA permeability test is based on the passive diffusion of the target compound through the artificial membrane. PAMPA synthetic membrane has a lipid-oil-lipid sandwich structure built into the pores of the porous filter. The middle oil layer maintains a strong and stable PAMPA membrane, is ultra-thin to minimize compound retention and interference with compound penetration. The test compound diluted in the buffer is placed in the donor well. The compound enters the artificial membrane from the donor well, entering the acceptor well by passive diffusion. (Kansy et al., Drug Discov Today Technol, 1 (4): 349-55 (2004); Avdeef, Expert Opin Drug Metab Toxicol., 1 (2): 325-42 (2005); Kerns et al., J Pharm Sci., 93 (6): 1440-53 (2004)). Pe is utilized to determine the rate of permeation. A larger absolute value of Pe in PAMPA indicates better permeability in vitro. A detailed protocol of PAMPA is described in Example 1.

The permeation enhancers disclosed herein can effectively enhance permeability of an epinephrine composition. In some embodiments, the absolute value of Pe of the pharmaceutical composition described herein in a PAMPA test is higher than 3×10⁻⁶ cm/s.

In addition, when the pharmaceutical composition is administered to a subject, maximum serum concentration of the active ingredient of the pharmaceutical composition (Cₘₐₓ) and the time to achieve Cₘₐₓ (Tₘₐₓ) are the two effective indexes for permeability in vivo. A larger Cₘₐₓ indicates a better permeability in vivo. A smaller Tₘₐₓ indicates a better permeability in vivo.

### Pharmacokinetics Profile

Compared to the permeation enhancer disclosed in the prior art i.e., C12E9, n-dodecyl-β-D-maltoside (DDM) and diethylene glycol monoethyl ether (DEGEE), the ones disclosed herein showed more desirable pharmacokinetics profiles. In some embodiments, the pharmaceutical compositions disclosed herein demonstrated better bioavailability. In some embodiments, the pharmaceutical compositions disclosed herein showed faster absorption rate, i.e., shorter Tₘₐₓ. In some embodiments, the pharmaceutical compositions disclosed herein showed larger early partial AUC.

The present disclosure also provides an unexpected finding that pharmaceutical compositions described herein showed higher bioavailability compared to the permeation enhancer disclosed in the prior art (such as C12E9, DDM and DEGEE), especially in the case of higher epinephrine dose, which is closer to clinically used dose. Some pharmaceutical compositions described herein even showed much higher bioavailability. For example, nasal delivery of an epinephrine composition comprising C12E7 as permeation enhancer showed 2.7 times higher exposure compared to epinephrine IM delivery.

Compared to compositions disclosed in the prior art (such as C12E9, DDM and DEGEE), much shorter Tₘₐₓ and larger early partial AUC were observed for the ones disclosed herein. Shorter Tₘₐₓ and larger early partial AUC are more favorable characters, because epinephrine is life-saving drug used in emergency situation and faster action of epinephrine is highly desirable (Assessment report of Neffy, EMA/204348/2022, Committee for Medicinal Products for Human Use, 25 Mar. 2022).

### Safety

Compared to the permeation enhancer disclosed in the prior art (such as C12E9, DDM and DEGEE), the ones disclosed herein have a better performance in terms of safety. In some embodiments, the pharmaceutical compositions disclosed herein demonstrated better tolerance. In some embodiments, the pharmaceutical compositions disclosed herein does not cause irreversible damage to nasal mucosa.

Epinephrine is a drug with narrow therapeutic window. A dose higher than the therapeutic window can potentially cause fatal side effects such as cerebrovascular hemorrhage, subarachnoid hemorrhage etc. Therefore, an ideal permeation enhancer shall increase the tolerance to epinephrine. In the disclosed PK studies, all subjects in the C12E9 group died after receiving nasal administration of epinephrine composition with C12E9 (1 mg/kg dose of epinephrine), with only 53.2% bioavailability compared with IM route. However, only 1 subject was in shock condition (recovered after cardiopulmonary resuscitation) after receiving nasal administration of epinephrine composition with C12E7 (1 mg/kg dose of epinephrine), with more than 270% bioavailability compared with IM route. By contrast, subjects became significantly less active after receiving nasal administration of 1 mg/kg dose epinephrine composition with C12E23 and epinephrine composition with C16E10, suggesting certain extent of adverse effect occurred. Subjects also remained normal and active after receiving nasal administration of epinephrine composition with C12E8, C12E4 or C10E6 (1 mg/kg dose of epinephrine), and much higher F, shorter Tₘₐₓ and larger early partial AUC were observed. Observations above indicates that C12E7, C12E8, C12E4 and C10E6, surprisingly, can increase the tolerance to epinephrine. C12E7, C12E8, C12E4 and C10E6, when added into an epinephrine composition, can increase a subject's tolerance to the composition. In some embodiments, the epinephrine composition is administered to the subject via nasal delivery route.

For the purpose of this application, a subject having better “tolerance” to a drug or drug composition means that the subject has less adverse effect, less severe adverse effect, or is less likely to have adverse effect after being administered with a compositions containing the drug or drug composition (such as epinephrine or epinephrine composition). Tolerance describes the ability of a subject to endure an active ingredient (for example, epinephrine) in large doses.

As used herein, irreversible damage refers to damage that cannot be restored spontaneously within relatively short amount of time. Irreversible damage can become more severe when permeation enhancers were used together epinephrine due to strong local pharmacological effect of epinephrine. For the purpose of this application, an insulin absorption test is used to evaluate nasal mucosa damage and determine whether the damage is irreversible or not (See Arnold, John J., et al., “Reestablishment of the nasal permeability barrier to several peptides following exposure to the absorption enhancer tetradecyl-β-D-maltoside,” Journal of Pharmaceutical Sciences, 99.4 (2010): 1912-1920). Under basal conditions, the nasal epithelium severely limits the absorption of drugs with molecular sizes larger than 1 kDa. Therefore, insulin, having a molecular size more than 5 kDa, generally cannot cross nasal mucosa, unless the nasal mucosa is damaged. By intranasally administering insulin after permeation enhancer's nasally exposure to permeation enhancer and epinephrine, one can determine whether the composition causes any damage to the subject's nasal mucosa, and whether the damage, if any, is reversible or not, by monitoring the Cₘₐₓ and AUC of insulin after nasally dosing insulin to the subject at various time after nasally dosing composition containing drug, i.e., epinephrine. Lower level Cₘₐₓ and AUC of insulin exposure indicates little damage to the nasal mucosa while higher level Cₘₐₓ and AUC of insulin exposure indicates nasal mucosa damage. In the case of reversible nasal mucosa damage, the Cₘₐₓ and AUC of insulin are high at first, then drops quickly, i.e., within about 2 hours, back to a significantly lower level. In the case of irreversible nasal mucosa damage, the Cₘₐₓ and AUC of insulin maintain at a higher level for a time longer than 2 hours. A detailed protocol of the insulin absorption test is set forth in Example 5.

The present disclosure also provides an unexpected finding that the pharmaceutical compositions described herein show much faster nasal mucosa damage reverse at a low pH, which is a pH value lower than 7, such as ranging from 4-6. The pharmaceutical compositions described herein cause only reversible damages, which restored much more quickly, when the pharmaceutical compositions are formulated to achieve a pH value of being lower than 7. In contrast, many permeation enhancers known in the art and used in nasal formulations, such as DDM or DEGEE do not show such pH sensitive trend regarding nasal mucosa damage as disclosed in Example 5.

Further, the pharmaceutical compositions described herein have better stability and permeability at a low pH, which is a pH value of being lower than 7, such as ranging from 4 to 6.

Compared with the compositions disclosed in the prior art (such as compositions containing permeation enhancers such as C12E9, DDM, DEGEE), it is also found that, surprisingly, current composition showed much less damage to nasal mucosa at a low pH, which is a pH value of being lower than 7, such as ranging from 4 to 6. For example, C12E9 has been reported to cause severe nasal cavity membrane damages when dosed with epinephrine via nasal route in a composition at pH 7.4 (Bleske et al., Effect of Vehicle on the Nasal Absorption of Epinephrine during Cardiopulmonary Resuscitation, Pharmacotherapy, 16 (6), 1039-1045 (1996)). However, the present disclosure provides that C12E9 causes much less damage at a low pH, which is a pH value of being lower than 7, such as ranging from 4 to 6.

In some embodiments, the pharmaceutical composition described herein has a pH of being lower than 7. In some embodiments, the pharmaceutical composition described herein has a pH ranging from 4 to 6. In some embodiments, the pharmaceutical composition described herein has a pH of 4, 5, or 6.

The present disclosure also provides a pharmaceutical composition comprising epinephrine and C12E9 as permeation enhancer, wherein the pH value of the pharmaceutical composition is lower than 7, such as ranging from 4 to 6.

### Stability

In some embodiments, the pharmaceutical composition described herein further comprising at least one pharmaceutically acceptable excipient, wherein the pharmaceutically acceptable excipient is a pH adjustment reagent, an antioxidant, a preservative, or an osmotic pressure regulator.

pH adjustment reagents, sometimes also written as pH modifiers, or pH adjusters, are substances used to adjust the pH of a pharmaceutical composition to a given range. pH is an expression of hydrogen ion concentration in water. Specifically, pH is the negative logarithm of hydrogen ion (H⁺) concentration (mol/L) in an aqueous solution: pH=−log₁₀(H⁺). In some embodiments, the pH adjustment reagent is an acid or a base. Some exemplary pH adjustment reagents include hydrochloric acid, acetic acid, phosphoric acid, sodium hydroxide, and ammonia.

Antioxidants are compounds that inhibit oxidation. In some embodiments, the antioxidant is used to improve stability of a pharmaceutical composition by delaying the oxidation of active substances and other excipients. Some exemplary antioxidant excipients include cysteine (CYS), sodium metabisulfite (SMB), propyl gallate (PG), butylated hydroxytoluene (BHT), D-α-tocopheryl polyethylene glycol succinate (vitamine E TPGS), ascorbic acid (VC), methionine, sodium hydrogen sulfite, sodium metabisulfite, sodium sulfite, alpha lipoic acid and butylated hydroxyanisole (BHA) (Celestino et al., Brazilian J. Pharma. Sci. 43-3, 405-415 (2012)).

Preservatives are substances added to a pharmaceutical composition to prevent undesirable physical, chemical, or biological changes. In some embodiments, the preservative is a bactericide or antimicrobial. Some exemplary preservatives include benzylkonium chloride, 2-trichloromethyl-2-propanol, butyl paraben, propyl paraben, benzethonium chloride, chlorocresol, phenol, and benzoic acid.

Osmotic pressure regulators are usually water-soluble substances with small molecular size. Osmosis is the diffusion of water across a membrane in response to osmotic pressure caused by an imbalance of molecules on either side of the membrane. Osmotic pressure regulators can change the osmotic pressure across cell membranes. Some exemplary osmotic pressure regulators include sodium chloride, gluose, mannitol, sorbital, lactose, phosphate acid, and citrate acid. In some embodiments, the pharmaceutical composition described herein comprises an antioxidant. In some embodiments, the antioxidant is selected from sodium hydrogen sulfite, sodium metabisulfite (SMB), propyl gallate (PG), sodium sulfite, ascorbic acid (VC), methionine, alpha lipoic acid, cysteine (CYS), D-α-tocopheryl polyethylene glycol succinate (vitamine E TPGS), butylated hydroxytoluene (BHT), and butyl hydroxyanisole (BHA). In some embodiments, the antioxidant is selected from sodium hydrogen sulfite and sodium metabisulfite. In some embodiments, the pharmaceutical composition comprises 0.2% sodium hydrogen sulfite. As shown in Example 4, an epinephrine formulation with C10E6, C12E4, C12E7, C12E8 and C12E9 as permeation enhancers show significantly better color stability profile when sodium hydrogen sulfite (as antioxidant) was used together with EDTA-2Na (as chelating agent). The pharmaceutical compositions with permeation enhancers and antioxidant have better stability and permeability at a low pH, which is a pH value of being lower than 7, such as ranging from 4 to 6.

As used herein, “stability” in the context of pharmaceutical composition refers to the ability of a pharmaceutical composition to retain its chemical, physical and biopharmaceutical properties over time. For the purpose of this disclosure, stability is indexed by “content percent” of the active ingredient in a composition. As used herein, content percent of an active ingredient at Day n is calculated by dividing the active ingredient's amount on Day n by the active ingredient's amount on Day 0, wherein the composition is stored under a given environmental condition (pH, temperature) starting from Day 0 and throughout the stress testing period, and wherein content percent is in weight percent.

In some embodiments, the content percent of the pharmaceutical composition described herein at pH 4.0, 60° C. is no lower than 90% by weight at day 30.

### Methods

In another aspect, the present disclosure provides a method comprising administering an effective amount of the pharmaceutical composition described herein to the subject. The route of administration is, for example, intravenous, intratumoral, oral, rectal, vaginal, parenteral, topical, pulmonary, intranasal, buccal, and ophthalmic.

In some embodiments, the method is used to treat a subject with type I allergic reaction. Type I allergic reaction is IgE-mediated immune response, which involves release of antibodies against the soluble antigen. This results in mast cell degranulation and release of histamine and other inflammatory mediators. Type I hypersensitivities include atopic diseases, which are an exaggerated IgE mediated immune responses (for example, rhinitis, conjunctivitis, and dermatitis), and allergic diseases, which are immune responses to foreign allergens (for example, anaphylaxis, urticaria, angioedema, and food and drug allergies).

In some embodiments, the allergic reaction is selected from allergic asthma, allergic conjunctivitis, allergic rhinitis, anaphylaxis, angioedema, urticaria, eosinophilia, drug allergy, and food allergy.

In some embodiments, the pharmaceutical composition is administered to the subject via nasal delivery.

As used herein, the term “Cₘₐₓ” refers to the maximum value of blood concentration shown on the curve that represents changes in blood concentrations of an active pharmaceutical ingredient (e.g., epinephrine), or a metabolite of the active pharmaceutical ingredient, over time. It would be understood that a person skilled in the art is able to select the suitable methods and conditions for measuring serum concentration of epinephrine in a subject and determining the Cₘₐₓ. It would be understood that a person skilled in the art is able to select the suitable methods and conditions for measuring serum concentration of epinephrine in a subject and determining the Tₘₐₓ.

As used herein, AUC refers to area under the curve, which is the area under the curve defined by changes in the blood concentration of an active ingredient (e.g., epinephrine), or a metabolite of the active ingredient, over time following the administration of a dose of the active ingredient. “AUC_{0-∞}” is the area under the concentration-time curve extrapolated to infinity following the administration of a dose. “AUC_{0-∞}” is the area under the concentration-time curve from time zero to time t following the administration of a dose, wherein t is the last time point with a measurable concentration.

As used herein, bioavailability (F) describes the percentage of an administered dose of a drug that reaches the systemic circulation. Its meaning is as defined in 21 C.F.R. § 320.1(a). For the purpose of the present disclosure, F specifically indicates the ratio of exposure per unit dose between nasal route and intramuscular route. For instance, F(AUC₀₋ₜ)=(AUC_{0-t, Intranasal}/Dose_{Intranasal})/(AUC_{0-t, Intramuscular}/Dose_{Intramuscular})×100%.

As used herein, early partial bioavailability (F₀₋₁₀) specifically indicates the ratio of AUC within the first 10 minutes after dosing per unit dose between nasal route and intramuscular route. For instance, F(AUC₀₋₁₀)=(AUC_{0-10, Intranasal}/Dose_{Intranasal})/(AUC_{0-10, Intramuscular}/Dose_{Intramuscular})×100%.

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1 Evaluation of Permeability In Vitro with PAMPA

96-well PAMPA kit (96-well skin PAMPA Sandwich set, PION Inc., MA, U.S.A.) was used to evaluate the impact of different permeability enhancers on epinephrine's permeability in vitro.

PRISMA™ buffer were prepared by diluting 25 mL of PRISMA™ (P/N 110151, PION Inc., MA, U.S.A.) with ultra-pure water into IL, followed by pH adjustment to 4.0 with 0.5 M NaOH solution.

A volume of 200 μL Hydration solution (PION Inc., MA, U.S.A.) was added into each well of support plate (P/N 110660, PION Inc., MA, U.S.A.). Precoated PAMPA plate (P/N 120657, PION Inc., MA, U.S.A.) was then submerged into Hydration solution overnight to allow sufficient hydration.

Permeation enhancers (concentration listed in Table 2) were dissolved in pH 4.0 aqueous buffer together with epinephrine (10 mg/mL, 50 mg/mL, 3 mg/mL) to make designed compositions; pH 4.0 epinephrine aqueous solution without permeation enhancers as reference composition. A volume of 200 μL of compositions were added into each well of doner plate; 200 μL of PRISMA™ buffer was added into each well of accepter plate. PAMPA plate was then put between donor plate and accepter plate to initiate permeation test. Permeation test for each composition was repeated in 4 wells. The PAMPA kit, including doner plate, PAMPA plate and acceptor plate, was covered and incubated at 37° C. for 5 hours. Antioxidants were also used to prevent oxidation during test. After incubation, the epinephrine concentration of doner plate and acceptor plate were determined using UPLC with the method described in Table 1.

TABLE 1
UPLC analytical method for epinephrine permeability
evaluation in vitro using PAMPA

| Mobile phase | Mobile | 0.05% Sodium heptane sulfonate | |
|---|---|---|---|
| | phase A | aqueous solution (Adjust pH to 2.4 with | |
| | | phosphoric acid) | |
| | Mobile | Mobile phase A: acetonitrile (1:1, v:v) | |
| | phase B | | |
| Gradient | Time | | |
| (isocratic) | (min) | Phase A % | Phase B % |
| | 0 | 90 | 10 |
| | 10.0 | 90 | 10 |

| | |
|---|---|
| Flow rate | 0.3 mL/min |
| Column type | Agilent ZORBAX Eclipse Plus-C18 |
| | (1.8 μm 2.1 mm*50 mm) |

Polyoxyethylene alkyl ethers containing double bonds in the alkyl chain below are written as C18-1E10 and C18-1E20. PAMPA test results for epinephrine solution with different types of permeation enhancers (0.1%, 0.25%, 2.5% w/v or v/v) is shown in Table 2. Compared with negative control (epinephrine without permeation enhancer, which showed only 1.1% permeated into acceptor well and 0.45×10⁻⁶ cm/s Pe), epinephrine solution with 0.1%-2.5% C10E6, C12E7, C12E8, C12E9, C16E10 all showed significantly and consistently higher epinephrine concentration in acceptor well as well as higher Pe values, i.e. more than 8% epinephrine permeated into acceptor well and Pe values higher than 3×10⁻⁶ cm/s. Compared with DEGEE, all PAMPA in vitro permeability test showed significant higher permeability when 0.1%-2.5% C10E6, C12E7, C12E8, C12E9, C16E10 are used; some permeation enhancers showed higher permeability than DDM, especially at lower permeation enhancer concentrations, which are more clinically relevant.

TABLE 2
In vitro permeability test results for epinephrine solution
with different types of permeation enhancers

| | | | Mean Percentage | | |
|---|---|---|---|---|---|
| | Proportion of | Epinephrine | of epinephrine in | | |
| Permeation | permeation | concentration | acceptor well | Pe (×10⁻⁶) | |
| enhancer used | enhancer | (mg/mL) | after incubation | cm/s | LogPe |
| Not used (negative | NA | 10 | 1.1% | 0.45 | −6.34 |
| reference) | | | | | |
| DDM (positive | 0.1% (w/v) | 50 | 7.2% | 2.78 | −5.56 |
| reference) | 0.25% (w/v) | 10 | 12.5% | 4.76 | −5.32 |
| | 2.5% (w/v) | 3 | 26.3% | 5.43 | −5.27 |
| C8E4 | 0.1% (v/v) | 50 | 4.8% | 2.26 | −5.65 |
| | 0.25%(v/v) | 10 | 2.8% | 1.00 | −6.00 |
| | 2.5%(v/v) | 3 | 42.4% | 5.19 | −5.28 |
| C10E6 | 0.1%(v/v) | 50 | 14.5% | 18.81 | −4.73 |
| | 0.25%(v/v) | 10 | 12.6% | 5.02 | −5.30 |
| | 2.5%(v/v) | 3 | 9.3% | 1.42 | −5.85 |
| C12E1 | 0.1%(v/v) | 50 | 1.4% | 0.45 | −6.35 |
| | 0.25%(v/v) | 10 | 0.4% | 0.15 | −6.82 |
| | 2.5%(v/v) | 3 | 0.7% | 0.21 | −6.68 |
| C12E4 | 0.1%(v/v) | 50 | 0.4% | 0.18 | −6.82 |
| | 0.25%(v/v) | 10 | 3.0% | 1.25 | −6.22 |
| | 2.5%(v/v) | 3 | 2.1% | 0.81 | −6.09 |
| C12E7 | 0.1%(v/v) | 50 | 10.8% | 5.47 | −5.27 |
| | 0.25%(v/v) | 10 | 8.2% | 3.23 | −5.54 |
| | 2.5%(v/v) | 3 | 11.9% | 3.17 | −5.58 |
| C12E8 | 0.1% (w/v) | 50 | 14.0% | 8.92 | −5.05 |
| | 0.25% (w/v) | 10 | 17.2% | 6.95 | −5.16 |
| | 2.5% (w/v) | 3 | 20.4% | 5.17 | −5.29 |
| C12E9 | 0.1%(v/v) | 50 | 13.8% | 6.97 | −5.16 |
| | 0.25%(v/v) | 10 | 9.2% | 3.81 | −5.68 |
| | 2.5%(v/v) | 3 | 9.9% | 3.59 | −5.46 |
| C12E23 | 0.1% (w/v) | 50 | 2.5% | 1.28 | −5.89 |
| | 0.25% (w/v) | 10 | 7.3% | 3.25 | −5.49 |
| | 2.5% (w/v) | 3 | 10.1% | 4.82 | −5.32 |
| C16E2 | 0.1% (w/v) | 50 | 0.8% | 0.35 | −6.48 |
| | 0.25% (w/v) | 10 | 0.4% | 0.25 | −6.65 |
| | 2.5% (w/v) | 3 | 0.6% | 0.40 | −6.41 |
| C16E10 | 0.1% (w/v) | 50 | 10.6% | 5.73 | −5.25 |
| | 0.25% (w/v) | 10 | 14.8% | 6.50 | −5.19 |
| | 2.5% (w/v) | 3 | 15.7% | 4.58 | −5.55 |
| C18E2 | 0.1% (w/v) | 50 | 1.2% | 0.64 | −6.24 |
| | 0.25% (w/v) | 10 | 0.6% | 0.39 | −6.41 |
| | 2.5% (w/v) | 3 | 0.7% | 0.38 | −6.45 |
| C18E10 | 0.1% (w/v) | 50 | 1.3% | 0.57 | −6.25 |
| | 0.25% (w/v) | 10 | 3.2% | 1.34 | −5.90 |
| | 2.5% (w/v) | 3 | 11.45 | 6.60 | −5.18 |
| C18-1E10 | 0.1%(v/v) | 50 | 6.5% | 4.16 | −5.39 |
| | 0.25%(v/v) | 10 | 13.7% | 6.12 | −5.22 |
| | 2.5%(v/v) | 3 | 16.5% | 8.36 | −5.09 |
| C18-1E20 | 0.1% (w/v) | 50 | 2.7% | 1.19 | −5.39 |
| | 0.25% (w/v) | 10 | 6.0% | 2.60 | −5.61 |
| | 2.5% (w/v) | 3 | 13.2% | 3.91 | −5.41 |
| DEGEE | 0.1%(v/v) | 50 | 1.8% | 0.71 | −6.15 |
| | 1.0%(v/v) | 10 | 0.5% | 1.82 | −6.75 |
| | 2.5%(v/v) | 3 | 3.6% | 0.51 | −6.29 |
| TritonX-100 | 1.0%(v/v) | 10 | 7.2% | 3.14 | −5.54 |
| SDS | 1.0%(w/v) | 10 | 9.0% | 3.61 | −5.45 |
| Tween-80 | 1.0%(v/v) | 10 | 6.7% | 2.97 | −5.53 |

### Example 2 Pharmacokinetics (PK) Studies Followed by Nasal Administration of Compositions Containing Permeation Enhancers

Intranasal compositions were prepared as follow: aqueous solution with 0.25% permeation enhancers, 0.2% sodium hydrogen sulfite and 0.9% saline were firstly prepared; epinephrine was then dissolved to reach either 10 mg/mL or 3 mg/mL epinephrine concentration; all solutions were then adjusted to pH 4 and refrigerated. Liquid compositions containing epinephrine and permeation enhancers were then dosed into nasal cavity according to Table 3.

Compositions were dosed to the nasal cavity of Sprague Dawley (SD) rats followed by blood sampling into 1.5 mL polyethylene centrifuge tubes at 5 min, 10 min, 15 min, 30 min, 45 min, 60 min, 90 min, and 120 min, respectively, after composition dosing. Sodium metabisulfite aqueous solution 10% (w/v) was prepared and added into samples according to volume ratio of 9:1 to prevent oxidation. Samples were vortexed well and stored at ice-water bath to wait for the pretreatment. Samples were stored in −80° C. freezer if pretreatment and analysis were not carried out in the same day of PK study. In the case of frozen sample, samples were equilibrated into room temperature before further pretreatment. A volume of 20 μL plasma sample was transferred into a 1.5-mL polyethylene centrifuge tube, adding 180 μL internal standard solution (preparation method shown in Table 5) before 5 min vortexing and 5 min centrifuging at 10,000 rpm (4° C.). A volume of 130 μL supernatant was transferred to a 96-well plate, adding 130 μL ultra-pure water, following by 5 min vortex mixing and centrifuging at 4000 rpm (4° C.) for 5 min. Pretreated samples were analyzed using LC-MS/MS with methods listed in Table 4, Table 6, and Table 7.

TABLE 3
Experimental design for rat PK study of different compositions

| | Epinephrine | Dose | | | Blood sampling |
|---|---|---|---|---|---|
| Dose | concentration | volume | Dose | | time (min) after |
| (mg/kg) | (mg/mL) | (mL/kg) | frequency | Route | dosing |
| 0.1 | 1 | 0.1 | Single | Intramuscular | at 5, 10, 15, 20, |
| | | | | injection (IM) | 30, 45, 60, 90, |
| 1 | 10 | 0.1 | Single | Intranasal (IN) | 120 minutes, |
| 0.3 | 3 | 0.1 | | | respectively |

TABLE 4
HPLC analytical procedure for epinephrine

| Mobile phase | Mobile phase A | 0.1% formic acid in water |
|---|---|---|
| | Mobile phase B | acetonitrile |
| Gradient | Time (min) | Mobile phase B % |
| | 0 | 65 |
| | 7.0/6.0 | stop |

| | |
|---|---|
| Flow rate | 0.60 mL/min |
| Injection volume | 5 μL/2 μL |
| Column | 35° C. |
| Column type | YMC-Triart Diol-HILIC 100*4.6 mm, 3 μm |

TABLE 5
Internal standard solution preparation method procedure.
Internal standard solution containing testosterone
(used in LC-MS/MS with AB SCIEX Triple Quad API 4000)

| | |
|---|---|
| Testosterone stock | Testosterone was accurately weighed, dissolved in the appropriate |
| solution A | amount of DMSO, and prepared to 10 mM concentration of stock |
| | solution as IS-A (using correction factors). |
| Testosterone stock | IS-B (100 μM) was prepared by adding 10 μL IS-A to 990 μL |
| solution B | acetonitrile and mixing well. |
| Testosterone | IS-C (5 μM) was prepared by adding 50 μL IS-B to 950 μL |
| working liquid C | acetonitrile and mixing well. |

Internal standard solution of nadolol
(used in LC-MS/MS with AB SCIEX Triple Quad 6500⁺)

| | |
|---|---|
| Nadolol stock | Nadolol was accurately weighed, dissolved in the appropriate |
| solution A | amount of DMSO, and prepared to 10 mM concentration of stock |
| | solution as IS-A (using correction factors). |
| Nadolol stock | IS-B (100 μM) was prepared by adding 10 μL IS-A to 990 μL |
| solution B | acetonitrile and mixing well. |
| Nadolol stock | IS-C (1 μM) was prepared by adding 10 μL IS-B to 990 μL |
| solution C | acetonitrile and mixing well. |
| Nadolol working | IS-D (5 nM) was prepared by adding 5 μL IS-C to 995 μL |
| liquid D | acetonitrile and mixing well. |

TABLE 6
Mass spectrometry method for epinephrine determination

| Instrument model | AB SCIEX Triple Quad API 4000 LC-MS/MS | | | | | | | |
|---|---|---|---|---|---|---|---|---|
| Ion source | Ion Spray | | Dwell time | | Collision cell exit | | Temperature | |
| | Voltage (IS) | | | | potential (CXP) | | | |
| | 5500 | V | 200 | ms | 12 | V | 600° | C. |
| | Entrance | | Collision Gas | | Curtain Gas | | Ion Source | |
| | potential (EP) | | (CAD) | | | | Gas1 | |
| | 10 | V | 6 | Psi | 32 | Psi | 38 | Psi |

TABLE 7
Mass spectrometry method for epinephrine determination

| Instrument model | AB SCIEX Triple Quad 6500⁺ LC-MS/MS | | | | | | | |
|---|---|---|---|---|---|---|---|---|
| Ion source | Ion Spray | | Dwell time | | Collision cell exit | | Temperature | |
| | Voltage (IS) | | | | potential (CXP) | | | |
| | 5500 | V | 200 | ms | 11 | V | 550° | C. |
| | Entrance | | Collision Gas | | Curtain Gas | | Ion Source | |
| | potential (EP) | | (CAD) | | | | Gas1 | |
| | 10 | V | 10 | Psi | 32 | Psi | 45 | Psi |

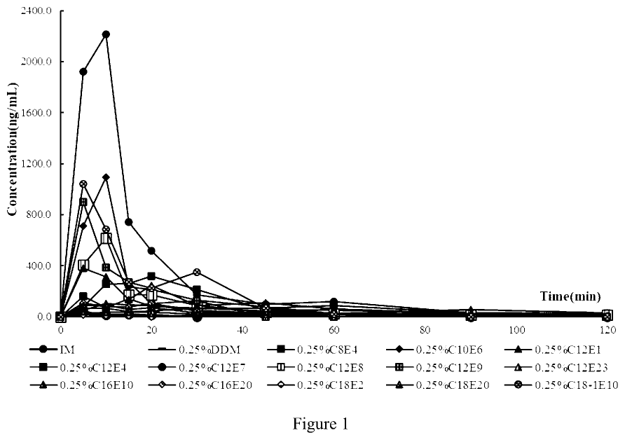

Epinephrine concentration in plasma versus time for 10 mg/mL epinephrine concentration (1 mg/kg dose) group is shown in Table 8 and FIG. 1. A number of 3 subjects (SD rats) were used for IM PK studies with epinephrine concentration of 1 mg/mL and 0.1 mg/kg dose. For intranasal route PK, 2 subjects (SD rats) were used with 10 mg/mL epinephrine concentration and 1 mg/kg dose. In Example 2 and Example 3, “NA” indicates that epinephrine was not detected at particular time points or parameters cannot be calculated due to no detection at particular time points. PK parameters, i.e., Tₘₐₓ, Cₘₐₓ, AUC, bioavailability (F₀₋ₜ) and early partial bioavailability within the first 10 minutes after dosing (F₀₋₁₀), were calculated using MaS Studio (v1.5.3.10) and shown in Table 9. It was observed that the Cₘₐₓ, AUC and bioavailability are substantially higher when polyoxyethylene alkyl ethers have 9-15 methylene groups and 4-10 oxyethylene units.

TABLE 8
Epinephrine concentration in plasma versus time of nasal dosed
compositions containing different permeation enhancers

| | dose | | | | | | | | | |
|---|---|---|---|---|---|---|---|---|---|---|
| | concentration | epinephrine concentration in plasma (ng/mL) versus time after dosing | | | | | | | | |
| Group | (mg/mL) | 5 min | 10 min | 15 min | 20 min | 30 min | 45 min | 60 min | 90 min | 120 min |
| Intramuscular | 1 | 38.6 ± | 15.7 ± | 16.9 ± | 13.1 ± | NA ± | 12.7 ± | 14.9 ± | NA ± | NA ± |
| injection | | 28.9 | NA | NA | NA | NA | NA | NA | NA | NA |
| (reference) | | | | | | | | | | |
| DDM | 10 | 64.5 ± | 64.3 ± | 42.1 ± | 33.3 ± | 16.4 ± | 26.1 ± | 59.0 ± | 16.7 ± | 30.2 ± |
| (0.25% w/v) | | 25.5 | 6.1 | 13.0 | 25.4 | 19.5 | 13.5 | 55.1 | NA | NA |
| C8E4 | 10 | 162.5 ± | 68.8 ± | 139.5 ± | 80.1 ± | 65.9 ± | 67.4 ± | 88.8 ± | 27.5 ± | 19.5 ± |
| (0.25% v/v) | | 18.5 | 37.3 | 21.3 | 17.0 | 5.8 | 52.1 | 34.6 | 7.2 | 11.4 |
| C10E6 | 10 | 710.6 ± | 1091 ± | 232.8 ± | 213.9 ± | 128.5 ± | 74.0 ± | 25.6 ± | 24.9 ± | 12.7 ± |
| (0.25% v/v) | | 354 | 343 | 76.2 | 147 | 65.0 | 40.7 | 17.3 | NA | 2.3 |
| C12E1 | 10 | 51.3 ± | 100.3 ± | 90.5 ± | 86.0 ± | 50.8 ± | 26.5 ± | 21.7 ± | 54.3 ± | 28.9 ± |
| (0.25% v/v) | | 72.6 | 22.0 | 80.3 | 31.8 | 49.0 | NA | NA | NA | NA |
| C12E4 | 10 | 78.1 ± | 254.4 ± | 257.8 ± | 315.6 ± | 211.1 ± | 49.9 ± | 48.7 ± | 27.9 ± | 23.1 ± |
| (0.25% v/v) | | 57.7 | 89.6 | 11.0 | 23.6 | 56.6 | 24.8 | 36.6 | 4.9 | 15.1 |
| C12E7 | 10 | 1922 ± | 2217 ± | 743.3 ± | 517.2 ± | 171.1 ± | 96.3 ± | 116.7 ± | 31.4 ± | 17.1 ± |
| (0.25% v/v) | | 330 | 118 | 83.3 | 324 | NA | NA | NA | NA | NA |
| C12E8 | 10 | 408.9 ± | 615.6 ± | 170.5 ± | 171.7 ± | 87.1 ± | 35.7 ± | 25.6 ± | 13.4 ± | 11.2 ± |
| (0.25% w/v) | | 184 | 124 | 136 | 5.5 | 1.8 | 1.5 | 10.9 | NA | NA |
| C12E9 | 10 | 897.8 ± | 386.7 ± | 271.1 ± | 104.2 ± | 122.2 ± | NA ± | NA ± | NA ± | NA ± |
| (0.25% v/v) | | 4.7 | NA | NA | NA | NA | NA | NA | NA | NA |
| C12E23 | 10 | 25.1 ± | 35.1 ± | 34.3 ± | 42.6 ± | 68.2 ± | 43.7 ± | 23.2 ± | 13.0 ± | 9.2 ± |
| (0.25% w/v) | | 0.8 | 15.8 | 15.5 | 17.9 | 26.5 | 32.3 | 2.3 | 2.3 | NA |
| C16E10 | 10 | 379.4 ± | 314.6 ± | 131.7 ± | 246.6 ± | 89.1 ± | 107.1 ± | 61.3 ± | 22.9 ± | NA ± |
| (0.25% w/v) | | 232 | 316 | 16.5 | 231 | 62.5 | NA | NA | NA | NA |
| C16E20 | 10 | 110.6 ± | 77.1 ± | 74.9 ± | 102.6 ± | 44.7 ± | 26.9 ± | 22.3 ± | NA ± | 1.6 ± |
| (0.25% w/v) | | 11.3 | 12.9 | 17.1 | 66.7 | 63.2 | 38.0 | 8.3 | NA | NA |
| C18E2 | 10 | 11.4 ± | 6.8 ± | 8.8 ± | NA ± | 11.4 ± | 11.4 ± | NA ± | NA ± | NA ± |
| (0.25% w/v) | | 7.6 | 9.6 | 12.5 | NA | NA | NA | NA | NA | NA |
| C18E20 | 10 | 86.9 ± | 91.0 ± | 54.3 ± | 70.3 ± | 29.1 ± | 33.5 ± | 18.3 ± | NA ± | 3.2 ± |
| (0.25% w/v) | | 111 | 69.3 | 49.0 | 75.0 | 6.1 | 16.9 | 5.7 | NA | NA |
| C18-1E10 | 10 | 1040 ± | 684.3 ± | 272.7 ± | 224.4 ± | 348.4 ± | 67.8 ± | 18.9 ± | NA ± | NA ± |
| (0.25% v/v) | | 604 | 92.5 | 76.1 | 108.4 | NA | NA | NA | NA | NA |

TABLE 9
PK parameters calculated from data listed in Table 8 (analytical method in Table 5 was
used for samples with *; analytical method in Table 6 was used for samples with †)

| | | PK Parameters | | | | |
|---|---|---|---|---|---|---|
| | | | | | | Early partial |
| Group | Dose | Tₘₐₓ | Cₘₐₓ | AUC₀₋ₜ | Bioavailability | bioavailability |
| (0.25%) | (mg/kg) | (min) | (ng/mL) | (min × ng/mL) | (F₀₋ₜ) | (F₀₋₁₀) |
| DDM* | 1 | 32.5 ± 38.9 | 90.2 ± 10.9 | 3355 ± 2159 | 31.3% | 20.8% |
| C8E4^{†} | 1 | 10 ± 7.1 | 165.1 ± 14.8 | 7407 ± 324 | 69.0% | 42.4% |
| C10E6^{†} | 1 | 10 ± 0 | 1091 ± 342 | 15815 ± 7080 | 147.4% | 270.4% |
| C12E1^{†} | 1 | 10 ± 7.1 | 125.0 ± 31.6 | 3644 ± 1538 | 34.0% | 21.8% |
| C12E4^{†} | 1 | 15 ± 7.1 | 325.0 ± 10.2 | 10984 ± 1477 | 102.4% | 44.2% |
| C12E7^{†} | 1 | 7.5 ± 3.54 | 2228 ± 102 | 30129 ± 6923 | 280.8% | 652.2% |
| C12E8* | 1 | 7.5 ± 3.54 | 621.1 ± 116 | 9982 ± 1162 | 93.0% | 154.2% |
| C12E9^{†} | 1 | 5.0 ± 0 | 897.8 ± 4.74 | 5703 ± 4880 | 53.2% | 234.8% |
| C12E23^{†} | 1 | 30 ± 0 | 68.2 ± 26.5 | 3194 ± 1189 | 29.8% | 9.2% |
| C16E10^{†} | 1 | 5.0 ± 0 | 379.5 ± 232 | 8588 ± 7927 | 80.0% | 115.5% |
| C16E20^{†} | 1 | 12.5 ± 0.6 | 134.2 ± 22.1 | 4074 ± 1139 | 38.0% | 32.1% |
| C18E2^{†} | 1 | 22.5 ± 10.6 | 14.5 ± 4.41 | 227 ± 44.2 | 2.1% | 3.2% |
| C18E20^{†} | 1 | 7.5 ± 3.5 | 103.8 ± 87.4 | 2953 ± 1714 | 27.5% | 28.5% |
| C18-1E10^{†} | 1 | 7.5 ± 3.54 | 1108.2 ± 507 | 13672 ± 1975 | 127.4% | 297.4% |
| IM^{†} | 0.1 | 5.0 ± 0 | 38.6 ± 28.9 | 1073 ± 1281 | NA | NA |

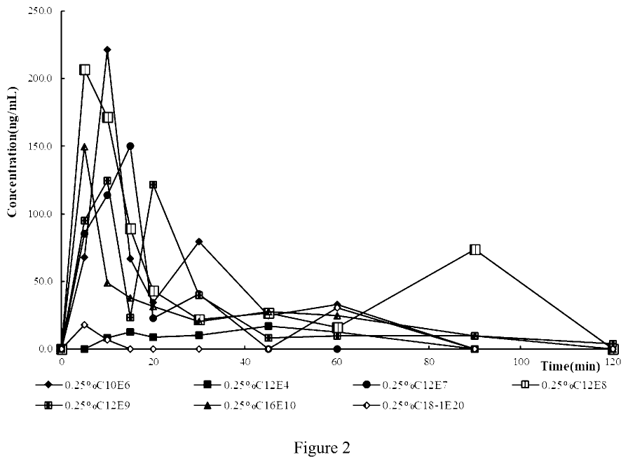

Epinephrine concentration in plasma versus time for 3 mg/mL epinephrine concentration (which is much lower than clinically used, 0.3 mg/kg dose) group is shown in Table 10 and FIG. 2. PK parameters, i.e., Tₘₐₓ, Cₘₐₓ, AUC and bioavailability, were calculated using MaS Studio (v1.5.3.10) and shown in Table 11. It was observed that Tₘₐₓ was much shorter while maintaining reasonably high Cₘₐₓ, AUC and bioavailability when polyoxyethylene alkyl ethers have 9-15 methylene groups and 4-10 oxyethylene units. Shorter Tₘₐₓ is a desirable from clinical perspective as epinephrine is needed to take action faster for treatment of type I allergic reaction.

TABLE 10
Epinephrine concentration in plasma versus time of nasal dosed
compositions containing different types of permeation enhancers

| | Epinephrine concentration in plasma (ng/mL) relative to time (n = 2) | | | | | | | | |
|---|---|---|---|---|---|---|---|---|---|
| Group | (epinephrine concentration: 3 mg/mL, dose 0.3 mg/kg) | | | | | | | | |
| (0.25% w/v) | 5 min | 10 min | 15 min | 20 min | 30 min | 45 min | 60 min | 90 min | 120 min |
| C10E6 | 67.8 ± | 222.1 ± | 66.7 ± | 34.3 ± | 79.6 ± | 24.7 ± | 32.8 ± | NA ± | NA ± |
| (0.25% v/v) | 19.6 | 47.1 | 67.6 | 31.7 | NA | NA | NA | NA | NA |
| C12E4 | NA ± | 8.2 ± | 12.4 ± | 8.9 ± | 10.1 ± | 17.1 ± | 12.4 ± | NA ± | NA ± |
| (0.25% v/v) | NA | NA | NA | NA | NA | NA | NA | NA | NA |
| C12E7 | 85.5 ± | 113.8 ± | 150.3 ± | 22.6 ± | 40.7 ± | NA ± | NA ± | NA ± | NA ± |
| (0.25% v/v) | 64.5 | 123.4 | 114.3 | 30.7 | NA | NA | NA | NA | NA |
| C12E8 | 206.7 ± | 171.6 ± | 89.0 ± | 43.3 ± | 21.8 ± | 26.5 ± | 16.1 ± | 73.7 ± | NA ± |
| (0.25% w/v) | 91.1 | 143.8 | 59.6 | 16.7 | 21.7 | 25.4 | 22.7 | NA | NA |
| C12E9 | 94.9 ± | 124.6 ± | 23.1 ± | 121.4 ± | 39.9 ± | 8.1 ± | 9.6 ± | 9.9 ± | 3.7 ± |
| (0.25% v/v) | 28.0 | 97.4 | 1.5 | 119.5 | 21.9 | 0.3 | NA | NA | NA |
| C16E10 | 149.8 ± | 48.7 ± | 38.0 ± | 31.6 ± | 20.6 ± | 27.4 ± | 24.7 ± | 9.7 ± | NA ± |
| (0.25% w/v) | 132.2 | 24.7 | 13.3 | 27.2 | NA | NA | NA | NA | NA |
| C18-1E20 | 18.1 ± | 6.6 ± | NA ± | NA ± | NA ± | NA ± | 30.5 ± | NA ± | NA ± |
| (0.25% w/v) | 11.4 | 9.3 | NA | NA | NA | NA | NA | NA | NA |

TABLE 11
PK parameters calculated from data listed in Table 10 (analytical method in Table 5 was
used for samples with *; analytical method in Table 6 was used for samples with^{†}).

| | PK Parameters (n = 2, dose concentration: 3 mg/mL) | | | | |
|---|---|---|---|---|---|
| | | | AUC₀₋ₜ | | Early partial |
| | | | (min × | Bioavailability | bioavailability |
| Group (0.25%) | Tₘₐₓ(min) | Cₘₐₓ(ng/mL) | ng/mL) | (F₀₋ₜ) | (F₀₋₁₀) |
| C10E6^{†} | 10.0 ± 0 | 222.1 ± 47.1 | 2811 ± 1619 | 87.3% | 127.9% |
| C12E4^{†} | 45.0 ± 0 | 34.2 ± NA | 1291 ± NA | 40.1% | 2.9% |
| C12E7^{†} | 15.0 ± 0 | 150.3 ± 114.3 | 1909 ± 1441 | 59.3% | 102.2% |
| C12E8^{†} | 7.5 ± 3.5 | 207.8 ± 92.6 | 4214 ± 3588 | 130.9% | 209.9% |
| C12E9^{†} | 15.0 ± 7.1 | 199.7 ± 8.8 | 3150 ± 647 | 97.9% | 112.7% |
| C16E10^{†} | 5.0 ± 0 | 149.8 ± 132.2 | 2202 ± 1156 | 68.4% | 125.0% |
| C18-1E20^{†} | 32.5 ± 38.9 | 20.3 ± 14.5 | 641 ± 871 | 19.9% | 15.3% |

### Example 3 Pharmacokinetics (PK) Profiles of Compositions with Different Permeation Enhancer Concentration and Epinephrine Concentrations

Intranasal compositions were prepared as follow: 0.1%, 0.25%, 1.0% and 2.5% permeation enhancers, 0.2% sodium hydrogen sulfite and 0.9% saline aqueous solutions were firstly prepared; epinephrine was then dissolved to reach either 10 mg/mL or 3 mg/mL epinephrine concentration (except 2.5% DEGEE composition, which had epinephrine concentration of 25 mg/mL); solutions were adjusted to pH 4 and refrigerated. Liquid compositions containing epinephrine and permeation enhancers were then dosed into nasal cavity according to Table 12.

Compositions were dosed to the nasal cavity of SD rats followed by blood sampling into 1.5 mL polyethylene centrifuge tubes at 5 min, 10 min, 15 min, 30 min, 45 min, 60 min, 90 min, and 120 min, respectively, after composition dosing. Prepare 10% (w/v) sodium metabisulfite aqueous solution, this solution was added into samples to prevent oxidation according to volume ratio of 9:1. Samples were vortexed well and stored at ice-water bath to wait for the pretreatment. Samples were stored in −80° C. freezer if pretreatment and analysis were not carried out in the same day. In the case of frozen sample, samples were equilibrated into room temperature before further pretreatment. A volume of 20 μL plasma sample was transferred into a 1.5-mL polyethylene centrifuge tube, adding 180 μL internal standard solution (preparation method is shown in Table 5) before 5 min vortexing and 5 min centrifuging at 10,000 rpm (4° C.). A volume of 130 μL supernatant was transferred to a 96-well plate, adding 130 μL ultra-pure water, following by 5 min vortex mixing and centrifuging at 4000 rpm (4° C.) for 5 min. Pretreated samples were analyzed using LC-MS/MS with methods listed in Table 4, Table 6, and Table 7.

TABLE 12
Experimental design for rat PK study

| | | Epinephrine | Dose | | | Blood sampling |
|---|---|---|---|---|---|---|
| | Dose | concentration | volume | Dose | | time (min) |
| Group | (mg/kg) | (mg/mL) | (mL/kg) | frequency | Route | after dosing |
| 0.1%(w/v) C12E8 | 1 | 10 | 0.1 | Single | Intranasal | at 5, 10, 15, |
| | | | | | (IN) | 20, 30, 45, |
| 0.25%(w/v) C12E8 | 1 | 10 | 0.1 | Single | Intranasal | 60, 90, |
| | | | | | (IN) | and120 |
| 1.0%(w/v) C12E8 | 1 | 10 | 0.1 | Single | Intranasal | minutes, |
| | | | | | (IN) | respectively |
| 2.5%(w/v) C12E8 | 0.3 | 10 | 0.1 | Single | Intranasal | |
| | | | | | (IN) | |
| 0.25%(w/v) DDM | 1 | 10 | 0.1 | Single | Intranasal | |
| | | | | | (IN) | |
| 0.25%(w/v) DDM | 2 | 20 | 0.1 | Single | Intranasal | |
| | | | | | (IN) | |
| 1.0%(w/v) DDM | 0.3 | 3 | 0.1 | Single | Intranasal | |
| | | | | | (IN) | |
| 1.0%(w/v) DDM | 1 | 10 | 0.1 | Single | Intranasal | |
| | | | | | (IN) | |
| 2.5%(w/v) DDM | 0.3 | 3 | 0.1 | Single | Intranasal | |
| | | | | | (IN) | |
| 1.0%(v/v) DEGEE | 2.5 | 25 | 0.1 | Single | Intranasal | |
| | | | | | (IN) | |
| 1.0%(v/v) TritonX- | 1 | 10 | 0.1 | Single | Intranasal | |
| 100 | | | | | (IN) | |
| 1.0%(w/v) SDS | 1 | 10 | 0.1 | Single | Intranasal | |
| | | | | | (IN) | |
| 1.0%(v/v) Tween-80 | 1 | 10 | 0.1 | Single | Intranasal | |
| | | | | | (IN) | |

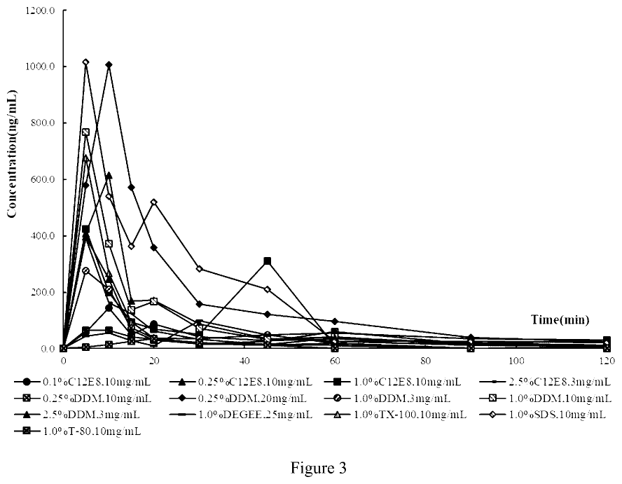

Epinephrine concentration in plasma at different time after dosing compositions with different epinephrine concentrations, permeation enhancer types and concentrations are shown in Table 13 and FIG. 3. It was observed that tested polyoxyethylene alkyl ether clearly showed permeation enhancement capacity in the concentration range from 0.1 mg/mL to 2.5 mg/mL. PK parameters, i.e., Tₘₐₓ, Cₘₐₓ, AUC, F₀₋ₜ and F₀₋₁₀, were calculated using MaS Studio (v1.5.3.10) and shown in Table 14. It was observed that the Cₘₐₓ, AUC and bioavailability generally increase with permeation enhancer concentration and epinephrine concentration when dose volume is fixed. Compared with groups containing DDM and DEGEE, with the same permeation enhancer and epinephrine concentration, polyoxyethylene alkyl ether consistently showed higher Cₘₐₓ, AUC and bioavailability, suggesting better permeation enhancement capacity. Shorter Tₘₐₓ was also observed for polyoxyethylene alkyl ether groups, suggesting faster for epinephrine to take effect.

TABLE 13
Epinephrine concentration in plasma at different time after dosing compositions with
different epinephrine concentration, permeation enhancer type and concentration

| | Dose | | | | | | | | | |
|---|---|---|---|---|---|---|---|---|---|---|
| | concentration | Epinephrine concentration in plasma (ng/mL) at different time after dosing | | | | | | | | |
| Group | (mg/mL) | 5 min | 10 min | 15 min | 20 min | 30 min | 45 min | 60 min | 90 min | 120 min |
| 0.1% | 10 (n = 3) | 57.7 ± | 143.8 ± | 49.6 ± | 86.3 ± | 40.8 ± | 28.4 ± | 35.4 ± | 19.9 ± | 11.3 ± |
| C12E8 | | 26.1 | 63.8 | 12.6 | 31.0 | 20.6 | 13.2 | NA | NA | NA |
| 0.25% | 10 (n = 2) | 408.9 ± | 615.6 ± | 170.5 ± | 171.7 ± | 87.1 ± | 35.7 ± | 25.6 ± | 13.4 ± | 11.2 ± |
| C12E8 | | 183.8 | 124.1 | 136.0 | 5.5 | 1.8 | 1.5 | 10.9 | NA | NA |
| 1.0% | 10 (n = 2) | 424.1 ± | 199.0 ± | 95.3 ± | 68.9 ± | 51.4 ± | 309.8 ± | 11.1 ± | NA ± | NA ± |
| C12E8 | | 337.4 | 44.0 | 3.2 | 50.5 | 24.1 | 399.6 | NA | NA | NA |
| 2.5% | 3 (n = 3) | 393.4 ± | 162.6 ± | 123.9 ± | 63.1 ± | 33.1 ± | 14.5 ± | 12.6 ± | NA ± | NA ± |
| C12E8 | | 111.4 | 58.2 | 74.4 | 27.0 | 3.2 | 5.0 | NA | NA | NA |
| 0.25% | 10 (n = 2) | 64.5 ± | 64.3 ± | 42.1 ± | 33.3 ± | 16.4 ± | 26.1 ± | 59.0 ± | 16.7 ± | 30.2 ± |
| DDM | | 25.5 | 6.1 | 13.0 | 25.4 | 19.5 | 13.5 | 55.1 | NA | NA |
| 0.25% | 20 (n = 2) | 580.0 ± | 1007 ± | 572.2 ± | 357.8 ± | 157.7 ± | 122.4 ± | 95.8 ± | 38.7 ± | 24.2 ± |
| DDM | | 198.0 | 1074 | 572.0 | 342.6 | 141.6 | 92.4 | 35.8 | 19.9 | 7.5 |
| 1.0% | 3 (n = 2) | 276.3 ± | 209.7 ± | 73.6 ± | 37.7 ± | 35.2 ± | 49.6 ± | 19.2 ± | NA ± | 6.3 ± |
| DDM | | 151.9 | 56.1 | 30.9 | 19.5 | 22.6 | NA | NA | NA | NA |
| 1.0% | 10 (n = 2) | 768.8 ± | 371.6 ± | 137.4 ± | 167.5 ± | 70.9 ± | 33.3 ± | 41.2 ± | 12.7 ± | 8.2 ± |
| DDM | | 864.1 | 394.6 | 92.2 | 177.4 | 58.7 | 18.6 | 30.5 | NA | NA |
| 2.5% | 3 (n = 3) | 396.1 ± | 246.9 ± | 64.2 ± | 30.0 ± | 17.7 ± | 11.6 ± | 19.5 ± | NA ± | NA ± |
| DDM | | 133.4 | 157.7 | 30.4 | 9.3 | 1.0 | 2.1 | NA | NA | NA |
| 1.0% | 25 (n = 2) | 44.5 ± | 56.5 ± | 29.2 ± | 10.5 ± | 97.7 ± | 47.8 ± | 56.3 ± | 35.3 ± | 30.1 ± |
| DEGEE | | 22.0 | 0.7 | 8.9 | 14.9 | 113.3 | 36.5 | 50.8 | 19.3 | 12.7 |
| 1.0% | 10 (n = 2) | 676.7 ± | 268.3 ± | 95.3 ± | 36.8 ± | 17.8 ± | 14.7 ± | 38.3 ± | 20.7 ± | NA ± |
| Triton | | 204.3 | 121.8 | 2.8 | 2.0 | 1.1 | 2.8 | 12.6 | NA | NA |
| X-100 | | | | | | | | | | |
| 1.0% | 10 (n = 2) | 1015.6 ± | 541.0 ± | 362.0 ± | 520.8 ± | 283.5 ± | 211.3 ± | 27.1 ± | 25.7 ± | 20.6 ± |
| SDS | | 1188 | 644.4 | 440.4 | 591.3 | 339.2 | 114.4 | NA | 25.6 | NA |
| 1.0% | 10 (n = 2) | 5.5 ± | 13.9 ± | 25.9 ± | 35.0 ± | 22.3 ± | 11.2 ± | NA ± | NA ± | NA ± |
| Tween- | | 7.8 | 0.3 | 16.6 | NA | NA | NA | NA | NA | NA |
| 80 | | | | | | | | | | |

TABLE 14
PK parameters calculated from data listed in Table 13

| | | PK Parameters | | | | |
|---|---|---|---|---|---|---|
| | Dose | | | | | Early partial |
| | concentration | Tₘₐₓ | Cₘₐₓ | AUC₀₋ₜ | Bioavailability | bioavailability |
| Group | (mg/mL) | (min) | (ng/mL) | (min × ng/mL) | (F₀₋ₜ) | (F₀₋₁₀) |
| 0.1% C12E8 | 10 (n = 3) | 10.0 ± 0 | 143.8 ± 63.8 | 3184 ± 269 | 29.7% | 27.9% |
| 0.25% C12E8 | 10 (n = 2) | 7.5 ± 3.5 | 621.1 ± 116.2 | 9982 ± 1162 | 93.0% | 154.2% |
| 1.0% C12E8 | 10 (n = 2) | 5.0 ± 0 | 424.1 ± 337.4 | 9482 ± 7811 | 88.4% | 112.7% |
| 2.5% C12E8 | 3 (n = 3) | 5.0 ± 0 | 393.4 ± 111.3 | 4482 ± 397 | 139.2% | 340.5% |
| 0.25% DDM | 10 (n = 2) | 32.5 ± 38.9 | 90.2 ± 10.9 | 3355 ± 2159 | 31.3% | 20.8% |
| 0.25% DDM | 20 (n = 2) | 7.5 ± 3.5 | 1103 ± 938 | 20967 ± 15266 | 97.7% | 116.6% |
| 1.0% DDM | 3 (n = 2) | 7.5 ± 3.5 | 276.9 ± 151.1 | 4174 ± 2526 | 129.7% | 273.4% |
| 1.0% DDM | 10 (n = 2) | 5.0 ± 0 | 768.8 ± 864.0 | 9739 ± 9435 | 90.8% | 205.4% |
| 2.5% DDM | 3 (n = 3) | 5.0 ± 0 | 396.1 ± 133.4 | 4247 ± 1633 | 131.9% | 372.7% |
| 1.0% DEGEE | 25 (n = 2) | 17.5 ± 17.7 | 118.9 ± 83.3 | 5445 ± 3827 | 20.3% | 6.3% |
| 1.0% | 10 (n = 2) | 5.0 ± 0 | 676.7 ± 204.3 | 6717 ± 2500 | 62.6% | 174.5% |
| TritonX-100 | | | | | | |
| 1.0% SDS | 10 (n = 2) | 5.0 ± 0 | 1016 ± 1188 | 22390 ± 21830 | 208.7% | 276.8% |
| 1.0% Tween- | 10 (n = 2) | 12.5 ± 3.5 | 25.9 ± 16.6 | 522 ± 510 | 4.9% | 2.7% |
| 80 | | | | | | |

### Example 4 Pharmacokinetics (PK) Studies Followed by Nasal Administration of Compositions Containing Permeation Enhancers

Intranasal compositions were prepared as follow: aqueous solution with 0.25% permeation enhancers, 0.2% sodium metabisulfite and 0.9% saline were firstly prepared; epinephrine was then dissolved to reach 2.5 mg/mL, 10 mg/mL and 20 mg/mL epinephrine concentration, respectively; all solutions were then adjusted to pH 4 and refrigerated before use. Liquid compositions containing epinephrine and permeation enhancers were then dosed into nasal cavity of beagle dogs according to Table 15.

Compositions were dosed to the nasal cavity of Beagle dogs followed by blood sampling at 1 min, 5 min, 10 min, 15 min, 30 min, 60 min, 90 min, and 120 min, respectively. Blood samples were collected into anticoagulation tubes with EDTA-K₂, followed by 10 min centrifuge under 1524 g, 4° C. Aliquot was then collected and stored under −40° C.˜−20° C. before analysis. For analysis, frozen aliquot samples were thawed and vortexed at ambient temperature. An aliquot of 100 μL of sample was added with 20 μL of water (contain 0.1% acetic acid, 50 ng/ml epinephrine-D6) as protein precipitation, followed by adding 250 μL PBA, vortexed well before adding 400 μL TOAB, the mixture was then vortexed for 10 min before being centrifuged at 13000 rpm for 5 min. 300 μL supernatant was then transferred into a 1.5 mL centrifuge tube, adding 200 μL n-caprylic alcohol and 125 μL 0.02N HCl solution, before 3-min vertexing. Then lower part solution was taken and mixed with 50 μL Sodium tetraborate buffer salt (100 mmol) and 100 μL benzoyl chloride (1%) before being vortexed for 0.5 min. An aliquot of 10 μL of the final mixture was injected into the LC-MS/MS system with methods listed in Table 16 and Table 7, respectively.

TABLE 15
Experimental design for dog PK study of different compositions

| | Epinephrine | Dose | | | Blood sampling |
|---|---|---|---|---|---|
| Dose | concentration | volume | Dose | | time (min) after |
| (mg/dog) | (mg/mL) | (mL/dog) | frequency | Route | dosing |
| 0.3 | 2.5 | 0.12 | Single | Intramuscular | at 1, 5, 10, 15, |
| | | | | injection (IM) | 20, 30, 60, 90, |
| 2 | 20 | 0.1 | Single | Intranasal (IN) | 120 minutes, |
| 1 | 10 | 0.1 | | | respectively |
| 2 | 20 | 0.1 | | | |

TABLE 16
HPLC analytical procedure for epinephrine

| Mobile phase | Mobile phase A | Water (0.1% formic acid, 5 mM |
|---|---|---|
| | | NH₄OAc) |
| | Mobile phase B | Acetonitrile (0.1% formic acid) |
| Gradient | Time (min) | Mobile phase B % |
| | 3.5 | 45 |
| | 6.0 | stop |

| | |
|---|---|
| Flow rate | 0.5 mL/min |
| Injection | 5 μL |
| volume | |
| Column | 30° C. |
| Column type | Agilent 300SB-C8 RRHD, 1.8 μm, 2.1 × 50 mm(50-521) |

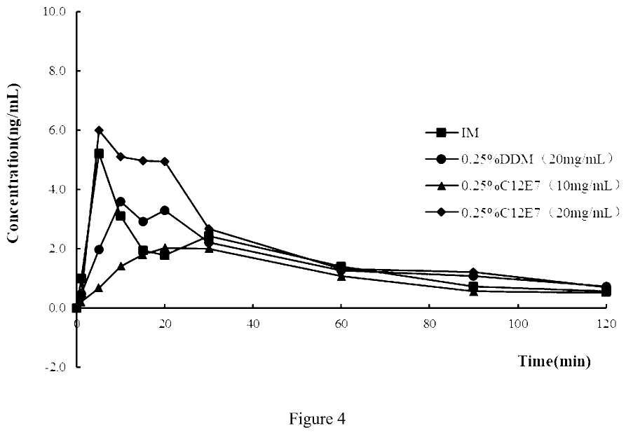

Table 17 and FIG. 4 showed the change of plasma epinephrine concentration over time in the prescription group with different dose concentrations and permeation enhancer types after administration at different time points. PK parameters, i.e., Tₘₐₓ, Cₘₐₓ, AUC, bioavailability relative to intramuscular injection route (F₀₋ₜ) and early partial bioavailability within the first 10 minutes after dosing (F₀₋₁₀), were calculated using Phoenix WinNonlin 8.3 software. C12E7 group showed higher bioavailability compared with commercial formulation with DDM as permeation enhancer, even at much lower epinephrine concentration and dose. C12E7 group also showed almost 3 times higher early partial AUC compared with DDM group (commercial formulation currently used). Higher early partial AUC is highly desirable when epinephrine is used in emergency situation for treating type I allergic reaction, including anaphylaxis . . .

TABLE 17
Epinephrine concentration in plasma versus time of nasal dosed
compositions containing different permeation enhancers

| | Dose | | | | | | | | | |
|---|---|---|---|---|---|---|---|---|---|---|
| | concentration | Epinephrine concentration in plasma (ng/mL) versus time after dosing | | | | | | | | |
| Group | (mg/mL) | 1 min | 5 min | 10 min | 15 min | 20 min | 30 min | 60 min | 90 min | 120 min |
| Intramuscular | 2.5 (n = 2) | 0.987 ± | 5.21 ± | 3.09 ± | 1.94 ± | 1.77 ± | 2.43 ± | 1.40 ± | 0.733 ± | 0.571 ± |
| injection | | 0.29 | 3.10 | 2.29 | 1.09 | 0.99 | 0.76 | 0.14 | 0.19 | 0.14 |
| (reference) | | | | | | | | | | |
| 0.25%(w/v) | 20 (n = 2) | 0.469 ± | 1.97 ± | 3.60 ± | 2.90 ± | 3.29 ± | 2.21 ± | 1.27 ± | 1.08 ± | 0.73 ± |
| DDM | | 0.33 | 0.06 | 1.15 | 0.67 | 1.56 | 1.43 | 0.06 | 0.23 | 0.02 |
| 0.25%(v/v) | 10 (n = 2) | 0.198 ± | 0.674 ± | 1.40 ± | 1.79 ± | 2.02 ± | 2.00 ± | 1.06 ± | 0.549 ± | 0.516 ± |
| C12E7 | | 0.01 | 0.23 | 0.40 | 0.94 | 1.80 | 2.04 | 0.70 | 0.21 | 0.03 |
| 0.25%(v/v) | 20 (n = 2) | 0.383 ± | 6.00 ± | 5.10 ± | 4.98 ± | 4.94 ± | 2.66 ± | 1.32 ± | 1.20 ± | 0.701 ± |
| C12E7 | | 0.28 | 6.09 | 3.85 | 3.12 | 2.28 | 0.03 | 0.06 | 0.69 | 0.27 |

TABLE 18
PK parameters calculated from data listed in Table 17

| | | PK Parameters | | | | |
|---|---|---|---|---|---|---|
| | | | | | | Early partial |
| | Dose | Tₘₐₓ | Cₘₐₓ | AUC₀₋ₜ | Bioavailability | bioavailability |
| Group | (mg/mL) | (min) | (ng/mL) | (min × ng/mL) | (F₀₋ₜ) | (F₀₋₁₀) |
| Intramuscular | 2.5 (n = 2) | 7.50 ± | 6.06 ± | 185.4 ± | NA | NA |
| injection | | 3.56 | 1.90 | 21 | | |
| (reference) | | | | | | |
| 0.25%(w/v) | 20 (n = 2) | 10.02 ± | 3.60 ± | 192.6 ± | 19.1% | 8.37% |
| DDM | | 0 | 1.15 | 58.8 | | |
| 0.25%(v/v) | 10 (n = 2) | 20.00 ± | 2.28 ± | 130.8 ± | 23.1% | 6.10% |
| C12E7 | | 14.13 | 1.64 | 89.4 | | |
| 0.25%(v/v) | 20 (n = 2) | 12.48 ± | 6.81 ± | 255 ± | 24.4% | 18.98% |
| C12E7 | | 10.61 | 4.94 | 102 | | |

### Example 5 Evaluation of Composition Stability

Intranasal compositions with 10 mg/mL epinephrine concentration were prepared by dissolving epinephrine in aqueous solution containing 0.25% different permeation enhancers and 0.9% sodium chloride. To prepare the formulation within an antioxidant and a preservative, epinephrine was dissolved in aqueous solution containing 0.1%˜1.0% different permeation enhancers, 0.2% antioxidant, 0.1% preservative and 0.9% sodium chloride. To prepare formulation within an antioxidant under different pH, epinephrine was dissolved in aqueous solution containing 1.0% different permeation enhancers, 0.2% antioxidant and 0.9% sodium chloride, with pH adjusted to 4.0, 5.0 and 6.0, and 7.0, respectively. For composition containing 1.0% C12E9, pH 7.4 samples were also prepared with 0.2% antioxidant and phosphate buffered (PBS) saline. Composition stability test design is shown in Table 19.

TABLE 19
Experimental design for formulation stability test

| | | Epinephrine | | |
|---|---|---|---|---|
| | Formulation | concentration | Stressing | |
| Group | pH | (mg/mL) | Temperature | Sampling time point |
| 0.25% different permeation enhancer | 4.0 | 10 | 60° C. | 0, 3, 7, 14, 21, 30 days |
| 0.1%, 0.25%, 1.0% permeation enhancer | 4.0 | 10 | 60° C. | 0, 3, 7, 14, 21, 30 days |
| with antioxidant and preservative | | | | |
| 1.0% permeation enhancer with | 4.0, 5.0, 6.0, | 10 | 60° C. | 0, 3, 7, 21, 30 days |
| antioxidant at different pH | 7.0, 7.4 | | | |

The liquid compositions were then divided into vials before capping and transferring into ovens for stressing. The content of epinephrine in different compositions before and after stressing were quantified by HPLC according to United States Pharmacopeia method (USP 35 <391>EPINEPHRINE ASSAY) as listed in Table 20. Phosphate buffer (pH 2.8) was prepared as follow: 5.0 g/L of potassium dihydrogen phosphate and 2.6 g/L of sodium octanesulfonate in water, followed by pH adjustment to 2.8; phosphate buffer passed through 0.45-μm filter before usage.

TABLE 20
HPLC Analytical procedure for epinephrine

| Mobile phase | Mobile phase A | Acetonitrile and pH 2.8 | |
|---|---|---|---|
| | | phosphate buffer (5:95) | |
| | Mobile phase B | Acetonitrile and pH 2.8 | |
| | | phosphate buffer (45:55) | |
| Gradient | Time (min) | Phase A % | Phase B % |
| | 0 | 95 | 5 |
| | 20 | 50 | 50 |
| | 21 | 50 | 50 |
| | 23 | 95 | 5 |
| | 30 | 95 | 5 |

| | |
|---|---|
| Flow rate | 1.2 mL/min |
| Column | 50° C. |
| Detector | UV 210 nm |
| Sample solution | Mobile phase A: 0.1M HCl = 90:10 |
| Column type | Waters Atlantis T3 3 um C18 100*4.6 mm |

The stability test results for compositions containing epinephrine and different permeation enhancers after 60° C. stressing for various time is shown in Table 21. It was observed that all formulations showed significant epinephrine content decrease after stressing. C12E9 and C16E2 showed worse stability profiles compared with other compositions. The rest compositions showed comparable stability profiles.

TABLE 21
The change of epinephrine content (%) for compositions containing epinephrine
and different permeation enhancers after 60° C. stressing

| | Content percent (stressing under 60° C.) | | | | | |
|---|---|---|---|---|---|---|
| | (epinephrine concentration: 10 mg/mL) | | | | | Appearance after |
| Group | 3 days | 7 days | 14 days | 21 days | 30 days | stressing |
| without permeation | 94.6% | 90.2% | 85.1% | 80.4% | 76.7% | All stressed |
| enhancer | | | | | | composition |
| DDM (0.25% w/v) | 92.8% | 86.4% | 84.6% | 79.8% | 75.7% | changed from clear |
| C10E6 (0.25% v/v) | 94.2% | 88.5% | 84.5% | 80.9% | 76.2% | at day 0 into red at |
| C12E4 (0.25% v/v) | 91.8% | 86.8% | 84.6% | 81.8% | 75.7% | day 3, then dark |
| C12E7 (0.25% v/v) | 93.0% | 85.7% | 80.5% | 80.2% | 75.9% | brown color |
| C12E8 (0.25% w/v) | 91.5% | 86.0% | 84.1% | 77.5% | 76.7% | quickly |
| C12E9 (0.25% v/v) | 90.5% | 86.2% | 83.4% | 76.3% | 71.9% | |
| C12E23 (0.25% w/v) | 93.4% | 89.7% | 84.7% | 81.0% | 77.1% | |
| C16E2 (0.25% w/v) | 93.4% | 89.3% | 84.5% | 82.0% | 69.9% | |
| C16E10 (0.25% w/v) | 92.9% | 89.2% | 84.0% | 77.3% | 72.0% | |
| C18E10 (0.25% w/v) | 93.4% | 86.0% | 84.9% | 80.2% | 75.2% | |

The stability test results (stressed under 60° C. for various time) for compositions with 10 mg/mL epinephrine, 0.25% C12E8 or DDM 60° C. with antioxidant, chelating agent and preservative are shown in Table 22. It was observed that sodium hydrogen sulfite significantly improved composition stability; composition appearance also remained clear for much longer time when both sodium hydrogen sulfite and EDTA-2Na were used together. Composition without sodium hydrogen sulfite showed red to dark brown color after stressing.

TABLE 22
The change of epinephrine content (%) while antioxidant, chelating
agent and preservative were used after stressing under 60° C.

| | (0.2% w/v) | | | | | | | | |
|---|---|---|---|---|---|---|---|---|---|
| | sodium | | (0.04% w/v) | Content percent (stressing under 60° C.) | | | | | Appearance after |
| Group | hydrogen | (0.2% w/v) | benzalkonium | (epinephrine concentration: 10 mg/mL) | | | | | stressing under |
| (0.25% w/v) | sulfite | EDTA-2Na | chloride | 3 days | 7 days | 15 days | 21 days | 30 days | 60° C. at day 30 |
| DDM | | ✓ | ✓ | 89.2% | 81.4% | 77.6% | 72.8% | 68.0% | dark |
| C12E8 | | | | 90.3% | 88.4% | 84.7% | 82.8% | 77.4% | brown |
| | ✓ | | | 94.7% | 92.8% | 94.1% | 90.8% | 90.9% | red |
| | | ✓ | | 90.2% | 81.2% | 80.0% | 71.8% | 64.5% | dark |
| | | | ✓ | 93.1% | 89.1% | 84.8% | 80.6% | 80.2% | brown |
| | | ✓ | ✓ | 91.0% | 84.7% | 77.2% | 76.1% | 69.3% | |
| | ✓ | | ✓ | 97.2% | 95.2% | 97.8% | 91.7% | 85.7% | |
| | ✓ | ✓ | | 95.1% | 91.2% | 88.4% | 87.4% | 85.9% | clear |

The stability test results of composition with 10 mg/mL epinephrine, 0.1% or 1.0% different permeation enhancers, 0.2% different antioxidants, 0.1% different preservatives after 60° C. stressing are shown in Table 23 and Table 24. No significant stability difference was found when both types of preservatives were added, indicating desirable chemical compatibility.

TABLE 23
The change of epinephrine content (%) for compositions containing different permeation enhancers
and preservatives (sodium hydrogen sulfite was used for all compositions, stressed under 60° C.)

| | Antioxidant: sodium hydrogen sulfite (0.2% w/v) | | | | | | | | | |
|---|---|---|---|---|---|---|---|---|---|---|
| Group | (Epinephrine concentration: 10 mg/mL) | | | | | | | | | |
| Preservative | 0.1% | 3 | 7 | 14 | 30 | 1% | 3 | 7 | 14 | 30 |
| (0.1% w/v) | CnEm | days | days | days | days | CnEm | days | days | days | days |
| NA | 0.1% | 96.1% | 95.6% | 94.3% | 92.5% | 1.0% | 96.7% | 95.7% | 95.2% | 94.4% |
| benzalkonium | (v/v) | 96.5% | 96.4% | 95.1% | 90.1% | (v/v) | 96.7% | 96.5% | 95.4% | 91.9% |
| chloride | C10E6 | | | | | C10E6 | | | | |
| 2-Trichloromethyl- | | 96.0% | 95.9% | 94.5% | 91.2% | | 96.3% | 96.2% | 95.4% | 93.8% |
| 2-propanol | | | | | | | | | | |
| NA | 0.1% | 96.2% | 95.4% | 94.7% | 93.5% | 1.0% | 95.9% | 95.8% | 94.0% | 93.6% |
| benzalkonium | (v/v) | 96.6% | 96.6% | 96.6% | 94.5% | (v/v) | 96.8% | 97.0% | 94.9% | 92.4% |
| chloride | C12E4 | | | | | C12E4 | | | | |
| 2-Trichloromethyl- | | 95.9% | 96.5% | 94.1% | 94.2% | | 95.9% | 95.7% | 94.1% | 93.0% |
| 2-propanol | | | | | | | | | | |
| NA | 0.1% | 96.1% | 96.1% | 95.4% | 93.8% | 1.0% | 96.6% | 96.2% | 94.8% | 91.6% |
| benzalkonium | (v/v) | 96.0% | 96.7% | 95.9% | 91.6% | (v/v) | 97.1% | 96.7% | 95.4% | 93.7% |
| chloride | C12E7 | | | | | C12E7 | | | | |
| 2-Trichloromethyl- | | 95.4% | 95.5% | 93.8% | 91.3% | | 96.0% | 95.80% | 94.50% | 90.5% |
| 2-propanol | | | | | | | | | | |
| NA | 0.1% | 96.7% | 96.5% | 95.2% | 91.2% | | 96.8% | 97.2% | 95.0% | 93.8% |
| benzalkonium | (w/v) | 96.2% | 96.4% | 95.9% | 93.7% | 1.0% | 98.1% | 97.4% | 94.5% | 89.0% |
| chloride | C12E8 | | | | | (w/v) | | | | |
| 2-Trichloromethyl- | | 97.2% | 96.3% | 95.4% | 91.2% | C12E8 | 96.8% | 97.0% | 95.3% | 93.5% |
| 2-propanol | | | | | | | | | | |
| NA | 0.1% | 96.3% | 96.9% | 96.5% | 83.6% | 1.0% | 98.8% | 96.4% | 95.4% | 91.1% |
| benzalkonium | (v/v) | 97.2% | 98.2% | 93.5% | 86.1% | (v/v) | 99.3% | 96.5% | 96.6% | 85.2% |
| chloride | C12E9 | | | | | C12E9 | | | | |
| 2-Trichloromethyl- | | 95.2% | 95.9% | 95.2% | 86.3% | | 99.2% | 97.1% | 94.9% | 88.5% |
| 2-propanol | | | | | | | | | | |

TABLE 24
The change of epinephrine content (%) for compositions containing different permeation enhancers
and preservatives (sodium metabisulfite was used for all compositions, stressed under 60° C.)

| | Antioxidant: sodium metabisulfite (0.2% w/v) | | | | | | | | | |
|---|---|---|---|---|---|---|---|---|---|---|
| Group | (Epinephrine concentration: 10 mg/mL) | | | | | | | | | |
| Preservative | 0.1% | 3 | 7 | 14 | 30 | 1% | 3 | 7 | 14 | 30 |
| (0.1% w/v) | CnEm | days | days | days | days | CnEm | days | days | days | days |
| NA | 0.1% | 97.5% | 96.5% | 95.4% | 94.3% | 1.0% | 96.4% | 95.8% | 94.6% | 94.1% |
| benzalkonium | (v/v) | 98.3% | 96.9% | 95.9% | 90.7% | (v/v) | 96.3% | 95.7% | 94.7% | 93.8% |
| chloride | C10E6 | | | | | C10E6 | | | | |
| 2-Trichloromethyl- | | 96.2% | 96.7% | 95.3% | 90.6% | | 96.1% | 95.0% | 92.9% | 93.0% |
| 2-propanol | | | | | | | | | | |
| NA | 0.1% | 95.2% | 94.4% | 92.5% | 93.3% | 1.0% | 96.2% | 95.2% | 93.6% | 94.6% |
| benzalkonium | (v/v) | 95.5% | 95.8% | 94.6% | 90.5% | (v/v) | 96.1% | 95.9% | 95.4% | 92.9% |
| chloride | C12E4 | | | | | C12E4 | | | | |
| 2-Trichloromethyl- | | 95.3% | 95.1% | 92.0% | 91.6% | | 95.7% | 94.7% | 92.7% | 92.4% |
| 2-propanol | | | | | | | | | | |
| NA | 0.1% | 94.6% | 94.2% | 92.5% | 92.7% | 1.0% | 96.2% | 95.2% | 93.8% | 90.1% |
| benzalkonium | (v/v) | 95.8% | 95.8% | 94.8% | 92.7% | (v/v) | 96.5% | 95.4% | 95.1% | 90.8% |
| chloride | C12E7 | | | | | C12E7 | | | | |
| 2-Trichloromethyl- | | 95.20% | 94.5% | 93.9% | 92.5% | | 96.8% | 95.7% | 93.8% | 91.0% |
| 2-propanol | | | | | | | | | | |
| NA | 0.1% | 97.3% | 96.8% | 95.7% | 91.1% | 1.0% | 96.6% | 95.1% | 94.7% | 90.2% |
| benzalkonium | (w/v) | 96.6% | 96.4% | 95.6% | 94.6% | (w/v) | 97.1% | 96.3% | 94.9% | 89.6% |
| chloride | C12E8 | | | | | C12E8 | | | | |
| 2-Trichloromethyl- | | 95.9% | 95.3% | 94.1% | 90.3% | | 96.6% | 95.2% | 93.6% | 92.5% |
| 2-propanol | | | | | | | | | | |
| NA | 0.1% | 95.9% | 95.9% | 93.7% | 94.8% | 1.0% | 98.3% | 94.2% | 94.1% | 84.4% |
| benzalkonium | (v/v) | 97.0% | 97.6% | 95.3% | 91.1% | (v/v) | 99.3% | 95.6% | 94.2% | 85.0% |
| chloride | C12E9 | | | | | C12E9 | | | | |
| 2-Trichloromethyl- | | 98.8% | 96.6% | 95.6% | 90.7% | | 98.9% | 95.7% | 94.8% | 85.4% |
| 2-propanol | | | | | | | | | | |

The stability test results of composition with 10 mg/mL epinephrine, 1.0% different permeation enhancers and 0.2% sodium hydrogen sulfite adjusted to pH 4.0, 5.0, 6.0 and 7.0 after 60° C. stressing is shown in Table 25. It was observed that sodium hydrogen sulfite significantly improved composition stability in pH range from 4.0 to 6.0, in which epinephrine contents remained higher than 95% after 7 days' stressing under 60° C. However, composition quickly became unstable when pH was equal or higher than 7.0. Moreover, it was found that sodium hydrogen sulfite only improved composition stability in the range of pH 4.0 and pH 6.0; when pH value was higher equal or higher than 7.0, adding sodium hydrogen sulfite caused even worse composition stability.

TABLE 25
The change of epinephrine content (%) of different formulation after stressing at 60° C.

| | | 0.2% sodium | Content percent (60° C.) | | | | |
|---|---|---|---|---|---|---|---|
| | | hydrogen | (epinephrine concentration: 10 mg/mL) | | | | Appearance after |
| Group | pH | sulfite | 3 days | 7 days | 21 days | 30 days | stressing |
| 1.0%(v/v) | pH 4.0 | | 92.2% | 91.7% | 81.2% | 78.4% | All stressed |
| C10E6 | | ✓ | 96.7% | 95.7% | 93.9% | 94.4% | composition with |
| | pH 5.0 | | 92.1% | 91.1% | 81.4% | 75.4% | antioxidant |
| | | ✓ | 96.9% | 97.2% | 96.3% | 87.5% | changed from |
| | pH 6.0 | | 93.2% | 90.9% | 82.5% | 76.8% | clear at day 3 and |
| | | ✓ | 96.5% | 97.1% | 95.3% | 91.6% | day 7 into dark |
| | pH 7.0 | | 93.1% | 90.7% | 79.9% | 75.6% | brown color at |
| | | ✓ | 75.8% | 70.7% | 61.9% | 62.1% | day 30, |
| 1.0%(v/v) | pH 4.0 | | 94.7% | 89.7% | 83.2% | 75.4% | composition |
| C12E4 | | ✓ | 95.9% | 95.8% | 93.8% | 93.6% | without |
| | pH 5.0 | | 93.4% | 90.2% | 84.2% | 74.6% | antioxidant |
| | | ✓ | 96.5% | 95.7% | 94.3% | 90.7% | changed from red |
| | pH 6.0 | | 92.7% | 90.1% | 82.2% | 73.6% | at day 3 into dark |
| | | ✓ | 95.3% | 96.2% | 94.3% | 90.7% | brown color at |
| | pH 7.0 | | 93.0% | 88.0% | 79.7% | 75.9% | day 30. |
| | | ✓ | 67.2% | 67.9% | 57.5% | 60.0% | |
| 1.0%(v/v) | pH 4.0 | | 94.2% | 89.9% | 81.4% | 78.2% | |
| C12E7 | | ✓ | 96.6% | 96.2% | 95.0% | 91.6% | |
| | pH 5.0 | | 93.4% | 89.0% | 83.5% | 75.0% | |
| | | ✓ | 96.7% | 97.1% | 95.4% | 93.5% | |
| | pH 6.0 | | 92.4% | 89.2% | 80.7% | 74.3% | |
| | | ✓ | 96.1% | 97.0% | 94.0% | 87.6% | |
| | pH 7.0 | | 90.1% | 81.9% | 79.5% | 77.3% | |
| | | ✓ | 70.0% | 69.9% | 56.9% | 60.5% | |
| 1.0%(w/v) | pH 4.0 | | 94.0% | 89.5% | 84.1% | 76.1% | |
| C12E8 | | ✓ | 96.8% | 97.2% | 95.2% | 93.8% | |
| | pH 5.0 | | 93.1% | 88.7% | 81.3% | 78.0% | |
| | | ✓ | 98.0% | 97.8% | 95.6% | 89.5% | |
| | pH 6.0 | | 91.5% | 89.0% | 81.1% | 83.2% | |
| | | ✓ | 96.5% | 97.1% | 95.1% | 91.2% | |
| | pH 7.0 | | 90.5% | 86.7% | 81.3% | 76.9% | |
| | | ✓ | 71.2% | 70.2% | 62.0% | 57.3% | |
| 1.0%(v/v) | pH 4.0 | | 92.3% | 91.6% | 83.5% | 75.9% | |
| C12E9 | | ✓ | 98.8% | 96.4% | 94.2% | 91.1% | |
| | pH 5.0 | | 91.9% | 89.3% | 81.7% | 76.3% | |
| | | ✓ | 96.4% | 97.0% | 95.0% | 93.8% | |
| | pH 6.0 | | 93.6% | 91.1% | 80.9% | 75.8% | |
| | | ✓ | 95.8% | 96.7% | 94.8% | 92.2% | |
| | pH 7.0 | | 92.7% | 88.1% | 78.8% | 77.1% | |
| | | ✓ | 70.3% | 69.0% | 58.1% | 57.6% | |
| | pH 7.4 | | 68.0% | 65.7% | 43.7% | 33.8% | |
| | | ✓ | 55.2% | 53.3% | 31.7% | 13.9% | |

### Example 6 Evaluation of Damage to Nasal Mucosa after Nasal Administrated Compositions Containing Epinephrine

As both permeation enhancers and epinephrine can potentially cause nasal mucosa damage, experiments were designed and carried to evaluate nasal mucosa damage after nasal dosing of compositions containing epinephrine and different permeation enhancers. Unless damaged, human insulin can hardly absorb through nasal mucosa. Therefore, as described in previous publication (See Arnold, John J., et al., “Reestablishment of the nasal permeability barrier to several peptides following exposure to the absorption enhancer tetradecyl-β-D-maltoside.” Journal of Pharmaceutical Sciences, 99.4 (2010): 1912-1920), insulin absorption though nasal mucosa at various time after nasal administrating of compositions containing permeations enhancers and epinephrine was used to evaluate nasal mucosa damage. High insulin absorption via nasal cavity indicates mucosa damage while no insulin absorption suggests intact nasal mucosa.

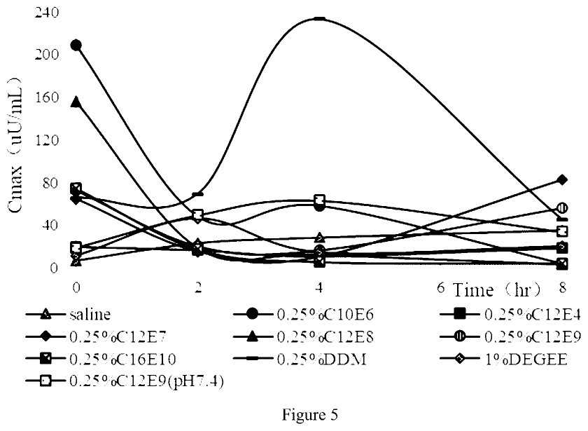

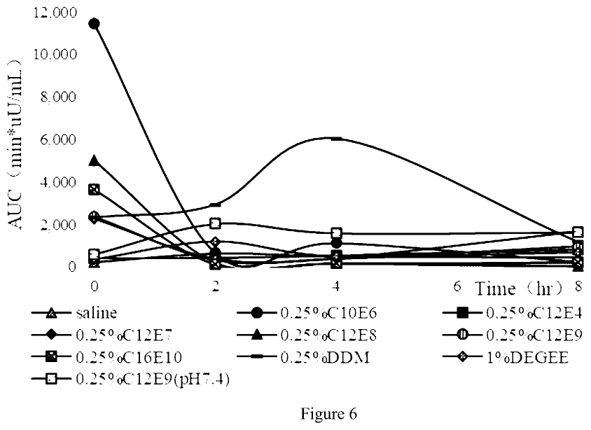

The evaluation process was conducted as follow: firstly, liquid compositions containing permeation enhancers and epinephrine were prepared according to Table 26; 0.5 μU/mL regular human insulin solution was also prepared separately; 8 subjects, i.e. healthy SD rats, were used to in each composition test group, with 2 subjects in each time point sub-group for each composition; each composition was then nasally dosed (0.1 mL/kg) to subjects in all corresponding 4 sub-groups listed in Table 22; for 0 hour sub-groups, regular human insulin (0.05 μU/kg dose, 0.5 μU/mL concentration, 0.1 mL/kg dose volume) was immediately administered to subjects' nasal cavity after epinephrine composition dosing; for 2 hour sub-groups, regular human insulin (0.05 μU/kg dose, 0.5 μU/mL concentration, 0.1 mL/kg dose volume) was administered to subjects' nasal cavity 2 hours after epinephrine composition dosing; for 4 hour sub-groups, regular human insulin (0.05 μU/kg dose, 0.5 μU/mL concentration, 0.1 mL/kg dose volume) was administered to subjects' nasal cavity 4 hours after epinephrine composition dosing; for 8 hour sub-groups, regular human insulin (0.05 μU/kg dose, 0.5 μU/mL concentration, 0.1 mL/kg dose volume) was administered to subjects' nasal cavity 8 hours after epinephrine composition dosing; blood samples were taken for all subjects at 0, 10, 20, 30, 45, 60, 90, 120 minutes after human insulin dosing; plasma insulin concentration was then determined using ELISA kit (Mercodia brand, operated according to instruction); PK parameters were then calculated using MaS Studio (v1.5.3.10) for each sub-group (Table 27, FIG. 5, and FIG. 6), thus reflecting nasal mucosa damage changes with time after different composition dosing.

TABLE 26
Experimental design for changes in nasal permeability

| | | | Nasal dose human | Blood sampling at 0, |
|---|---|---|---|---|
| | | Nasal dose | insulin (0.05 μU/kg | 10, 20, 30, 45, 60, |
| | | compositions | dose, 0.5 μU/mL | 90, 120 minute after |
| | | to 8 subjects | concentration, 0.1 | human insulin dosing |
| | | per | mL/kg dose volume) to | (determine plasma |
| | | composition | 2 subjects accordingly | insulin concentration |
| | | group (1 | to time designed for | by ELISA kit and |
| Composition groups (n = 2 | | mL/kg dose | each sub-group of | calculate corresponding |
| for each sub-group) | | volume); | composition group; | PK parameters) |
| Saline | 0 hour sub-group | ✓ | ✓ | ✓ |
| | 2 hour sub-group | | ✓ | ✓ |
| | 4 hour sub-group | | ✓ | ✓ |
| | 8 hour sub-group | | ✓ | ✓ |
| C10E6 (with 0.25% v/v | 0 hour sub-group | ✓ | ✓ | ✓ |
| C10E6 and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| C12E4(with 0.25% v/v | 0 hour sub-group | ✓ | ✓ | ✓ |
| C12E4 and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| C12E7(with 0.25% v/v | 0 hour sub-group | ✓ | ✓ | ✓ |
| C12E7 and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| C12E8(with 0.25% w/v | 0 hour sub-group | ✓ | ✓ | ✓ |
| C12E8 and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| C12E9(with 0.25% v/v | 0 hour sub-group | ✓ | ✓ | ✓ |
| C12E9 and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| C16E10 (with 0.25% w/v | 0 hour sub-group | ✓ | ✓ | ✓ |
| C16E10 and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| DDM(with 0.25% w/v | 0 hour sub-group | ✓ | ✓ | ✓ |
| DDM and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| DEGEE(with 1% v/v | 0 hour sub-group | ✓ | ✓ | ✓ |
| DEGEE and 3 mg/mL | 2 hour sub-group | | ✓ | ✓ |
| epinephrine, | 4 hour sub-group | | ✓ | ✓ |
| adjusted to pH 4.0) | 8 hour sub-group | | ✓ | ✓ |
| C12E9(with 0.25% v/v | 0 hour sub-group | ✓ | ✓ | ✓ |
| C12E9 and 3 mg/mL | 2 hour sub-group | | ✓ | ✓ |
| epinephrine, | 4 hour sub-group | | ✓ | ✓ |
| adjusted to pH 7.4) | 8 hour sub-group | | ✓ | ✓ |

Insulin PK parameters surprisingly showed that compositions with C12E9 showed irreversible damage at higher pH, e.g., pH 7.4; much lower and reversible damage was observed when the composition pH was lower, e.g., pH 4.0. All compositions with polyoxyethylene alkyl ether disclosed herein at lower pH showed fast (within 2 hours) mucosa recovery to normal state compared with observed irreversible nasal mucosa damage when DDM, DEGEE, and C12E9 (high pH) were used. Moreover, higher insulin AUC and Cₘₐₓ at 0 hour was also observed for tested compositions with polyoxyethylene alkyl ethers, indicating faster drug absorption has occurred compared with DDM, DEGEE, and C12E9 (high pH). Hence, especially when used under proper pH environment, polyoxyethylene alkyl ethers disclosed herein can be used as a much safer (e.g., faster nasal mucosa recovery) and more effective (e.g., faster drug absorption after application) permeation enhancer compared with other permeation enhancers disclosed in the prior art.

TABLE 27
Reversibility of permeability barrier to nasally administered insulin of different permeation enhancers

| | PK Parameters of insulin for each sub-group listed in Table 21 | | | | | |
|---|---|---|---|---|---|---|
| | (n = 2 for each sub-group) | | | | | |
| Composition | Sub- | Cₘₐₓ | AUC₀₋ₜ | Sub- | Cₘₐₓ | AUC₀₋ₜ |
| groups | groups | (μU/mL) | (min × μU/mL) | group | (μU/mL) | (min × μU/mL) |
| saline | 0 hour | 7.64 ± 0.25 | 215.12 ± 49.07 | 4 hour | 28.95 ± 38.42 | 498.54 ± 659.99 |
| | 2 hour | 23.83 ± 18.19 | 631.65 ± 449.80 | 8 hour | 35.68 ± 39.81 | 439.99 ± 568.95 |
| C10E6 (with | 0 hour | 209.08 ± 115.61 | 11453.44 ± 8112.76 | 4 hour | 58.76 ± 27.98 | 1132.26 ± 94.08 |
| 0.25% v/v | 2 hour | 48.91 ± 64.79 | 677.96 ± 789.04 | 8 hour | 4.61 ± 2.98 | 249.37 ± 98.03 |
| C10E6 and 3 | | | | | | |
| mg/mL | | | | | | |
| epinephrine, | | | | | | |
| adjusted to pH | | | | | | |
| 4.0) | | | | | | |
| C12E4 (with | 0 hour | 20.31 ± 17.08 | 390.61 ± 137.59 | 4 hour | 11.16 ± 0.44 | 530.24 ± 176.47 |
| 0.25% v/v | 2 hour | 17.11 ± 4.83 | 413.05 ± 147.99 | 8 hour | 19.13 ± 7.47 | 824.01 ± 194.85 |
| C12E4 and 3 | | | | | | |
| mg/mL | | | | | | |
| epinephrine, | | | | | | |
| adjusted to pH | | | | | | |
| 4.0) | | | | | | |
| C12E7 (with | 0 hour | 65.10 ± 27.63 | 2274.33 ± 1357.53 | 4 hour | 10.68 ± 8.49 | 404.48 ± 484.45 |
| 0.25% v/v | 2 hour | 15.77 ± 3.28 | 378.65 ± 208.27 | 8 hour | 82.84 ± 25.66 | 1663.07 ± 35.68 |
| C10E7 and 3 | | | | | | |
| mg/mL | | | | | | |
| epinephrine, | | | | | | |
| adjusted to pH | | | | | | |
| 4.0) | | | | | | |
| C12E8 (with | 0 hour | 155.76 ± 164.45 | 5010.13 ± 5427.36 | 4 hour | 13.11 ± 15.42 | 174.23 ± 210.58 |
| 0.25% w/v | 2 hour | 19.38 ± 9.64 | 223.58 ± 161.37 | 8 hour | 3.91 ± 2.81 | 51.18 ± 28.28 |
| adjusted to pH | | | | | | |
| 4.0) | | | | | | |
| C12E9 (with | 0 hour | 73.17 ± 53.73 | 2381.93 ± 1073.99 | 4 hour | 17.09 ± 13.79 | 371.29 ± 415.12 |
| 0.25% v/v | 2 hour | 20.96 ± 7.02 | 317.84 ± 336.41 | 8 hour | 56.62 ± 36.95 | 973.01 ± 385.76 |
| C12E9 and 3 | | | | | | |
| mg/mL | | | | | | |
| epinephrine, | | | | | | |
| adjusted to pH | | | | | | |
| 4.0) | | | | | | |
| C16E10 (with | 0 hour | 75.18 ± 39.76 | 3644.02 ± 2910.81 | 4 hour | 5.91 ± 0.50 | 162.94 ± 121.38 |
| 0.25% w/v | 2 hour | 17.78 ± 20.09 | 121.46 ± 69.42 | 8 hour | 4.44 ± 2.19 | 211.89 ± 22.05 |
| C16E10 and 3 | | | | | | |
| mg/mL | | | | | | |
| epinephrine, | | | | | | |
| adjusted to pH | | | | | | |
| 4.0) | | | | | | |
| DDM (with | 0 hour | 65.97 ± 9.01 | 2334.72 ± 815.98 | 4 hour | 233.53 ± 194.78 | 6035.65 ± 4935.82 |
| 0.25% w/v | 2 hour | 69.53 ± 12.67 | 2935.31 ± 2170.98 | 8 hour | 45.49 ± 21.91 | 1183.56 ± 404.92 |
| DDM and 3 | | | | | | |
| mg/mL | | | | | | |
| epinephrine, | | | | | | |
| adjusted to pH | | | | | | |
| 4.0) | | | | | | |
| DEGEE (with | 0 hour | 11.90 ± 11.99 | 357.90 ± 199.61 | 4 hour | 15.23 ± 12.77 | 502.49 ± 283.55 |
| 1.0% v/v | 2 hour | 46.88 ± 28.65 | 1190.31 ± 460.78 | 8 hour | 20.81 ± 24.02 | 683.78 ± 629.23 |
| DEGEE and 3 | | | | | | |
| mg/mL | | | | | | |
| epinephrine, | | | | | | |
| adjusted to pH | | | | | | |
| 4.0 | | | | | | |
| C12E9 (with | 0 hour | 19.38 ± 2.48 | 569.92 ± 214.62 | 4 hour | 63.43 ± 16.67 | 1590.71 ± 633.65 |
| 0.25% v/v | 2 hour | 49.84 ± 22.30 | 2037.69 ± 547.44 | 8 hour | 34.40 ± 12.15 | 1625.95 ± 985.46 |
| C12E9 and 3 | | | | | | |
| mg/mL | | | | | | |
| epinephrine, | | | | | | |
| adjusted to pH | | | | | | |
| 7.4) | | | | | | |

What is claimed is:

1. A pharmaceutical composition, comprising epinephrine and a permeation enhancer in the formula: CH₃(CH₂)ₙ₋₁[OCH₂CH₂]ₘOH,
wherein n is an integer selected from 10, 11, 12, 13, 14, 15, and 16, and m is an integer selected from 4, 5, 6, 7, 8 and 9.

2. The pharmaceutical composition of claim 1, wherein n is 12, and m is selected from 4, 7, and 8.

3. The pharmaceutical composition of claim 1, wherein n is 10, and m is 6.

4. The pharmaceutical composition of any one of claims 1-3, wherein the concentration of the permeation enhancer ranges from 0.1% to 2.50% (v/v).

5. The pharmaceutical composition of any one of claims 1-4, wherein the concentration of the permeation enhancer is 0.25% (v/v).

6. The pharmaceutical composition of any one of claims 1-5, wherein the pH value of the pharmaceutical composition is lower than 7.

7. The pharmaceutical composition of any one of claims 1-6, wherein the pH value of the pharmaceutical composition ranges from 4 to 6.

8. The pharmaceutical composition of any one of claims 1-7, wherein the pharmaceutical composition is used for nasal administration, or is used in a nasal delivery device.

9. The pharmaceutical composition of any one of claims 1-8 further comprising at least one pharmaceutically acceptable excipient, wherein the pharmaceutically acceptable excipient is a pH regulator, an antioxidant, a preservative, or an osmotic pressure regulator.

10. The pharmaceutical composition of claim 9, wherein the antioxidant is selected from sodium hydrogen sulfite, sodium metabisulfite (SMB), propyl gallate (PG), sodium sulfite, ascorbic acid (VC), methionine, alpha lipoic acid, cysteine (CYS), D-α-tocopheryl polyethylene glycol succinate (vitamine E TPGS), butylated hydroxytoluene (BHT) and butyl hydroxyanisole (BHA).

11. The pharmaceutical composition of claim 9 or 10, wherein the antioxidant is selected from sodium hydrogen sulfite and sodium metabisulfite.

12. The pharmaceutical composition of any one of claims 1-11 wherein the concentration of epinephrine in the pharmaceutical composition ranges from 0.3% to 5% (w/v).

13. The pharmaceutical composition of any one of claims 1-12, wherein the pharmaceutical composition has a dosage form of liquid or spray.

14. The pharmaceutical composition of any one of claims 1-13, wherein the recovery rate of the pharmaceutical composition at pH 4.0, at 60° C. is no lower than 90% by weight at day 30.

15. The pharmaceutical composition of any one of claims 1-14, wherein the absolute value of Pe of the pharmaceutical composition in a PAMPA test is higher than 3× 10⁻⁶ cm/s.

16. The pharmaceutical composition of any one of claims 1-15, wherein the pharmaceutical composition does not cause irreversible damage to the nasal mucosa.

17. A method comprising administering an effective amount of the pharmaceutical composition of any one of claims 1-16 to the subject.

18. The method of claim 17, wherein the method is used to treat a subject with type I allergic reaction.

19. The method of claim 17, wherein the method is used to treat conditions selected from allergic asthma, allergic conjunctivitis, allergic rhinitis, anaphylaxis, angioedema, urticaria, eosinophilia, drug allergy, and food allergy.

20. The method of any one of claims 17-19, wherein the pharmaceutical composition is administered to the subject via nasal delivery.
